(19) United States
(12) Patent Application Publication
(10) Pub. No.: US 2023/0225973 A1
(43) Pub. Date: Jul. 20, 2023

(54) MULTI-LAYERED MEDICAMENT FOR INHALATION

(71) Applicant: Mylan Pharma UK Limited, Hertfordshire (GB)

(72) Inventor: Gavin Magee, Kent (GB)

(73) Assignee: Mylan Pharma UK Limited, Hertfordshire (GB)

(21) Appl. No.: 18/011,770

(22) PCT Filed: Jun. 25, 2021
(86) PCT No.: PCT/IB2021/000444

§ 371 Date: Dec. 20, 2022

Related U.S. Application Data
(60) Provisional application No. 63/044,406, filed on Jun. 26, 2020.

(51) Int. Cl. A61K 9/14; A61P 11/00

(52) U.S. Cl. CPC A61K 9/14; A61P 11/00; A61M 2202/064

(57) ABSTRACT

A medicament for inhalation is provided. The medicament comprises at least a first layer comprising a first active pharmaceutical ingredient in powder form and at least a second layer comprising at least a second active pharmaceutical ingredient in powder form. The second layer is disposed on the first layer. A container for dispensing a medicament and methods of making are also described.

### CROSS REFERENCE TO RELATED APPLICATION(S)

This application claims priority to U.S. Provisional Application Ser. No. 63/044,406, filed on Jun. 26, 2020, the entire disclosure of which is hereby incorporated by reference in its entirety into the present disclosure.

### BACKGROUND

### Field

Development of inhalation technology for drug delivery has contributed immensely in treating various intrapulmonary and extrapulmonary diseases. This is supported by the lungs' unique geometry such as large surface area, thin alveolar epithelial lining, high vascularization, and avoidance of first-pass metabolism. Numerous inhalation delivery systems have been developed and studied to treat lung diseases such as asthma, chronic obstructive pulmonary disease (COPD), and other pulmonary infections. Among them, three approaches, that is, nebulizers, pressurized metered-dose inhalers (pMDIs), and dry powder inhalers (DPI), are extensively scrutinized for the treatment of several lung diseases and pathological conditions. Utilization of nebulizers requires bulky compressors or a source of compressed air, while pMDIs have limitations such as sedimentation, crystal growth, and selection of appropriate propellant and they emit dosages at high velocity, which build deposition in the oropharynx most commonly where they are swallowed and enhance the risk of systemic absorption. DPIs were introduced to cure some of the weaknesses associated with nebulizers and pMDIs. DPIs provide physicochemical stability and deep lungs deposition using the patient's respiration. Additionally, they do not require cold chain storage or reconstitution of powders into solutions for nebulization.

Layering of medicament formulations for oral delivery, (e.g., tablet form) has been used for delivering two active pharmaceutical ingredients to a patient. For example, bilayered tablets of atorvastatin and atenolol have been made that have an initial fast-release of atorvastatin in the stomach while providing sustained-release of atenolol after oral administration. However, when it comes to oral and nasal inhalation delivery systems and delivering two or more active pharmaceutical ingredients (APIs), the APIs are often blended together for inhalation. For example, inhalation products are typically administered in pockets or receptacles of dose units. In blended formulations, a pocket will contain a mixture of two or more APIs that are blended together in every pocket for inhalation. In the blended configuration of APIs, it may be difficult to maintain the individual performance characteristics of the medicament prior to administration.

At times, it is desirable to administer two or more APIs simultaneously. In this configuration, one API is separated from the other and provided as two separate dose units for inhalation within a single delivery device such as an inhaler. For example, a first pocket can contain a first API and a second pocket can contain a second API that is different from the first one, whereby the device (e.g., inhaler) can be used to deliver a combination of APIs stored in separate pockets. However, administration of APIs in two separate doses may not be beneficial, as the release and emptying rate of the API may be affected by the separate pockets.

Therefore, it would be beneficial to provide a medicament that comprises two or more layers, where each layer comprises a separate API that is disposed within the same unit dose receptacle of the inhalation delivery systems so that the APIs of the medicament are delivered simultaneously. It would also be beneficial if the layers do not substantially mix in the dose unit so that each API's individual performance characteristics are essentially maintained prior to administration of the medicament to the patient.

### SUMMARY

The present application provides a medicament for inhalation that comprises two or more layers, where each layer comprises a separate API that is disposed within the same unit dose receptacle of the inhalation delivery system. In some embodiments, the layers do not substantially mix in the dose unit so that each APIs' individual performance characteristics are essentially maintained prior to administration of the medicament to the patient.

In one non-limiting embodiment, a medicament for inhalation is provided. The medicament comprises at least a first layer comprising a first active pharmaceutical ingredient in powder form and at least a second layer comprising at least a second active pharmaceutical ingredient in powder form. The second layer is disposed on the first layer.

In another non-limiting embodiment, a container for dispensing a medicament for inhalation is provided. The medicament comprises at least a first layer comprising a first active pharmaceutical ingredient in powder form and at least a second layer comprising at least a second active pharmaceutical ingredient in powder form. The second layer is disposed on the first layer.

In yet another non-limiting embodiment, a method of making a medicament for inhalation is provided, the method comprising: providing a container comprising a pocket; and disposing the medicament into the pocket of the container, wherein the medicament comprises at least a first layer comprising a first active pharmaceutical ingredient in powder form and at least a second layer comprising at least a second active pharmaceutical ingredient in powder form, the second layer disposed on the first layer, wherein the second layer is disposed on the first layer in a linear or a non-linear configuration.

Various aspects of the present disclosure may be further characterized by one or more of the following clauses:

Clause 1: A medicament for inhalation, the medicament comprising at least a first layer comprising a first active pharmaceutical ingredient in powder form and at least a second layer comprising at least a second active pharmaceutical ingredient in powder form, the second layer disposed on the first layer.

Clause 2: The medicament according to clause 1, wherein the at least first layer further comprises a first excipient blended with the first active pharmaceutical ingredient to form a first dry powder and the at least second layer further comprises a second excipient blended with the at least second active pharmaceutical ingredient to form a second dry powder.

Clause 3: The medicament according to clause 2, wherein the first and second excipients are selected from the group consisting of glucose, arabinose, lactose, sucrose, maltose, dextrans, magnesium stearate, leucine, isoleucine, lysine, valine, methionine, phenylalanine, and combinations thereof.

Clause 4: The medicament according to any one of clauses 1-3, wherein the at least first layer and the at least second layer comprise micronized powder.

Clause 5: The medicament according to any one of clauses 1-4, wherein: (i) the at least second layer is disposed on the at least first layer in a linear configuration; (ii) the at least second layer is disposed on the at least first layer in a non-linear configuration; or (iii) the first active pharmaceutical ingredient and the at least second active pharmaceutical ingredient are different active pharmaceutical ingredients.

Clause 6: The medicament according to any one of clauses 1-5, wherein the at least first layer and the at least second layer mix at a boundary layer interface in an amount from about 0.01 to about 10 wt. %.

Clause 7: The medicament according to any one of clauses 1-6, wherein: (i) the first active pharmaceutical ingredient comprises an anticholinergic agent and the at least second active pharmaceutical ingredient comprises a bronchodilator; (ii) the first active pharmaceutical ingredient comprises an anticholinergic agent and the at least second active pharmaceutical ingredient comprises a bronchodilator and the medicament comprises a third active pharmaceutical ingredient comprising a corticosteroid; or (iii) the first active pharmaceutical ingredient comprises an anticholinergic agent in the first layer and the at least second active pharmaceutical ingredient comprises a bronchodilator and a corticosteroid in the second layer.

Clause 8: The medicament according to clause 7, wherein the anticholinergic agent comprises 5-[3-(3-Hydroxyphenoxy)azetidin-1-yl]-5-methyl-2,2-diphenylhexanamide hydrochloride, the bronchodilator comprises salmeterol xinafoate and the corticosteroid comprises fluticasone propionate.

Clause 9: The medicament according to clause 7 or clause 8, wherein the anticholinergic agent comprises the structure of

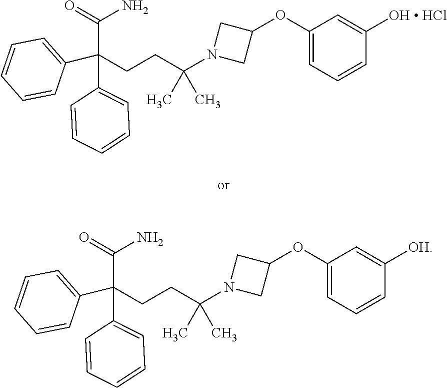

Clause 10: The medicament according to clause 8 or clause 9, wherein the 5-[3-(3-Hydroxyphenoxy)azetidin-1-yl]-5-methyl-2,2-diphenylhexanamide hydrochloride is in a dose of about 40 to about 800 mcg, the salmeterol xinafoate is in a dose of about 72.5 mcg, and the fluticasone propionate is in a dose of about 100 to about 500 mcg.

Clause 11: The medicament according to any one of clauses 8-10, wherein the 5-[3-(3-Hydroxyphenoxy)azetidin-1-yl]-5-methyl-2,2-diphenylhexanamide hydrochloride has an average particle size of from about 1 μm to about 10 μm, the salmeterol xinafoate has an average particle size of from about 1 μm to about 10 μm, and the fluticasone propionate has an average particle size from about 1 μm to about 3 μm.

Clause 12: A container for dispensing a medicament for inhalation, the container having one or more doses of the medicament, and the medicament comprising at least a first layer comprising a first active pharmaceutical ingredient in powder form and at least a second layer comprising at least a second active pharmaceutical ingredient in powder form, the second layer disposed on the first layer.

Clause 13: The container according to clause 12, wherein the medicament is used for oral inhalation.

Clause 14: The container according to clause 12 or clause 13, wherein the container comprises a pocket containing a dose of the medicament and a covering disposed on the container, the covering configured for piercing engagement such that the dose of the medicament can be dispensed from the container.

Clause 15: The container according to any one of clauses 12-14, wherein the at least first layer and the at least second layer mix at a boundary layer interface in an amount from about 0.01 to about 10 wt. %.

Clause 16: The container according to any one of clauses 12-15, wherein: (i) the first active pharmaceutical ingredient comprises an anticholinergic agent comprising 5-[3-(3-Hydroxyphenoxy)azetidin-1-yl]-5-methyl-2,2-diphenylhexanamide hydrochloride, the at least second active pharmaceutical ingredient comprises a bronchodilator comprising salmeterol xinafoate; (ii) the first active pharmaceutical ingredient comprises an anticholinergic agent comprising 5-[3-(3-Hydroxyphenoxy)azetidin-1-yl]-5-methyl-2,2-diphenylhexanamide hydrochloride, the at least second active pharmaceutical ingredient comprises a bronchodilator comprising salmeterol xinafoate and a corticosteroid comprising fluticasone propionate; or (iii) the first active pharmaceutical ingredient comprises an anticholinergic agent comprising 5-[3-(3-Hydroxyphenoxy)azetidin-1-yl]-5-methyl-2,2-diphenylhexanamide hydrochloride, the at least second active pharmaceutical ingredient comprises a bronchodilator comprising salmeterol xinafoate and the medicament further comprises a third layer comprising a third active pharmaceutical ingredient comprising a corticosteroid comprising fluticasone propionate.

Clause 17: The container according to clause 16, wherein the 5-[3-(3-Hydroxyphenoxy)azetidin-1-yl]-5-methyl-2,2-diphenylhexanamide hydrochloride is in a dose of about 40 to about 800 mcg, the salmeterol xinafoate is in a dose of about 72.5 mcg, and the fluticasone propionate is in a dose of about 100 to about 500 mcg.

Clause 18: The container according to any one of clauses 12-17, wherein: (i) the medicament has a density of about 0.6 to about 0.9 mg/μl; or (ii) the medicament comprises an excipient comprising glucose, arabinose, lactose, mannitol, sucrose, maltose, dextrans, magnesium stearate, leucine, isoleucine, lysine, valine, methionine, phenylalanine, or a combination thereof.

Clause 19: The container according to any one of clauses 14-18, wherein the pocket comprises a fill weight of the first active pharmaceutical ingredient and a first excipient that is in an amount of about 3 to about 9 mg, and a fill weight of the at least second active pharmaceutical ingredient and a second excipient that is in an amount of about 8 to about 13 mg.

Clause 20: The container according to any one of clauses 14-19, wherein the pocket comprises a fill weight of the first active pharmaceutical ingredient and a first excipient that is in an amount of about 7 mg, a fill weight of the at least second active pharmaceutical ingredient and a second excipient that is in an amount of about 7 mg and a fill weight of a third active pharmaceutical ingredient and a third excipient in an amount of about 7 mg.

Clause 21: A method of making a medicament for inhalation, the method comprising: adding the medicament into a container configured for oral or nasal inhalation, wherein the medicament comprises at least a first layer comprising a first active pharmaceutical ingredient in powder form and at least a second layer comprising at least a second active pharmaceutical ingredient in powder form, the at least second layer disposed on the at least first layer.

Clause 22: The method according to clause 21, wherein the at least second layer is disposed on the at least first layer in a non-linear configuration.

Clause 23: The method according to clause 21 or clause 22, wherein: (i) the at least first layer and the at least second layer are disposed and configured in the container via a filling head; (ii) the first active pharmaceutical ingredient comprises an anticholinergic agent comprising 5-[3-(3-Hydroxyphenoxy)azetidin-1-yl]-5-methyl-2,2-diphenylhexanamide hydrochloride, and the at least second active pharmaceutical ingredient comprises a bronchodilator comprising salmeterol xinafoate and a corticosteroid comprising fluticasone propionate; or (iii) the at least first layer further comprises a first excipient blended with the first active ingredient to form a first dry powder, and the at least second layer further comprises a second excipient blended with the at least second active pharmaceutical ingredient to form a second dry powder.

Clause 24: A medicament for inhalation, the medicament comprising at least a first layer comprising a first active pharmaceutical ingredient in powder form and at least a second layer comprising an excipient in powder form, the second layer disposed on the first layer.

Clause 25: The medicament according to any one of clauses 1-11, wherein the at least first layer further comprises a first excipient blended with the first active pharmaceutical ingredient to form a first dry powder.

Other features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWING

In part, other aspects, features, benefits and advantages of the embodiments will be apparent with regard to the following description, appended claims, and accompanying drawings in which:

FIGS. 1A-F illustrates a front view of an embodiment of a container for a medicament. The medicament comprises at least a first layer comprising a first active pharmaceutical ingredient in powder form and at least a second layer comprising at least a second active pharmaceutical ingredient in powder form. The second layer is disposed on the first layer. In some embodiments, the medicament comprises a third layer and/or a fourth layer. The layers are shown disposed in linear or non-linear configurations;

FIG. 2 illustrates embodiments of a medicament layering inside a pocket of a container;

FIG. 3 illustrates layering configurations within a container;

FIG. 4 illustrates a dry powder inhaler device that is configured for use with the medicament of FIGS. 1A-F;

FIG. 5 illustrates a front cross sectional view of a container for a medicament being filled with a first layer of a first active pharmaceutical ingredient and a second layer of at least a second active pharmaceutical ingredient;

FIG. 6 illustrates results of a comparison of fine particle dose performance of a blended combination medicament product versus a layered medicament as compared to a baseline; and

FIG. 7 illustrates results of a study that was done to determine whether two different layering configurations of a medicament had the same or different fill weight results. The medicament comprised of a layer of 5-[3-(3-Hydroxyphenoxy)azetidin-1-yl]-5-methyl-2,2-diphenylhexanamide hydrochloride and an excipient, and a layer of salmeterol xinafoate combined with fluticasone propionate and an excipient. When 7 mg of the combined 5-[3-(3-Hydroxyphenoxy)azetidin-1-yl]-5-methyl-2,2-diphenylhexanamide hydrochloride and excipient was added as the first layer, the standard deviation (SD) of the first layer was lower than when it was filled as the second layer. When 13 mg of the combined salmeterol xinafoate, fluticasone propionate and excipient was added as the first layer or the second layer, results showed that there was no impact on SD. The results indicated that filling the container with the lowest fill weight first, followed by the highest fill weight second, provided for a better control of fill weight.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

The present disclosure may be understood more readily by reference to the following detailed description of the disclosure presented in connection with the accompanying drawings, which together form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. The following description is presented to enable any person skilled in the art to make and use the present disclosure.

Notwithstanding that the numerical ranges and parameters setting forth the broad scope of the application are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

As used in the specification and including the appended claims, the singular forms “a,” “an,” and “the” include the plural, and reference to a particular numerical value includes at least that particular value, unless the context clearly dictates otherwise.

Ranges may be expressed herein as from “about” or “approximately” one particular value and/or to “about” or “approximately” another particular value. When such a range is expressed, another embodiment includes from the one particular value and/or to the other particular value.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, or the like, are also used to describe various elements, regions, sections, etc. and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features.

The term “pharmaceutically acceptable salt” or “salt” comprises inorganic and organic salts. Examples of organic salts may include formate, acetate, trifluoroacetate, propionate, butyrate, lactate, citrate, tartrate, malate, maleate, succinate, methanesulfonate, benzenesulfonate, xinafoate, pamoate, and benzoate. Examples of inorganic salts may include fluoride chloride, bromide, iodide, phosphate, nitrate and sulphate.

The term “medicament” includes a substance suitable for oral or nasal inhalation. The medicament can include an active pharmaceutical ingredient and an excipient.

As used herein, the term “active pharmaceutical ingredient” (API) as used herein, includes any substance (i.e., compound or composition of matter) which, when administered to an organism (human or animal) induces a desired pharmacologic and/or physiologic effect by local and/or systemic action. The term therefore encompasses substances traditionally regarded as actives, drugs or bioactive agents, as well as biopharmaceuticals (e.g., peptides, hormones, nucleic acids, gene constructs, etc.) typically employed to treat a number of conditions which is defined broadly to encompass diseases, disorders, infections, or the like. Exemplary APIs include, without limitation, antibiotics, antivirals, H₂-receptor antagonists, 5HT₁ agonists, 5HT₃ antagonists, COX2-inhibitors, steroids (e.g., prednisone, prednisolone, dexamethasone) APIs used in treating psychiatric conditions such as depression, anxiety, bipolar condition, tranquilizers, APIs used in treating metabolic conditions, anticancer APIs, APIs used in treating neurological conditions such as epilepsy and Parkinson's Disease, APIs used in treating cardiovascular conditions, non-steroidal anti-inflammatory APIs, APIs used in treating Central Nervous System conditions, or APIs employed in treating hepatitis. The APIs can be muscarinic M3 receptor agonists or anticholinergic agents, β2-adrenoceptor agonists, compounds having a dual muscarinic antagonist and β2-agonist activity and glucocorticoid receptor agonists.

In some embodiments, the API is ipratropium, tiotropium, oxitropium, trospium, aclidiniums, perenzepine, telenzepine, ephedrine, adrenaline, isoprenaline, metaproterenol, phenylephrine, phenylpropanolamine, pirbuterol, reproterol, rimiterol, isoetharine, carmoterol, albuterol, terbutaline, bambuterol, fenoterol, salbutamol, tulobuterol formoterol, salmeterol, prednisone, prednisolone, flunisolide, triamcinolone acetonide, beclomethasone, budesonide, fluticasone, ciclesonide, mometasone as well as salts and/or solvates thereof. In some embodiments, the API is glycopyrronium bromide, formoterol fumarate, tiotropium bromide. In some embodiments, the API is a glucocorticosteroid, such as, for example, fluticasone, budesonide, mometasone or ciclesonide.

In some embodiments, the active pharmaceutical ingredient is 5-[3-(3-Hydroxyphenoxy)azetidin-1-yl]-5-methyl-2,2-diphenylhexanamide hydrochloride, which is a long acting anticholinergic having the following structure of formula II:

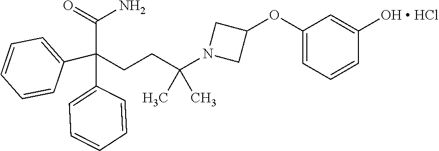

and having the molecular formula of C₂₈H₃₂N₂O₃·HCl.

In some embodiments, the active pharmaceutical ingredient is 5-[3-(3-Hydroxyphenoxy)azetidin-1-yl]-5-methyl-2,2-diphenylhexanamide, which is a long acting anticholinergic free base having the following structure:

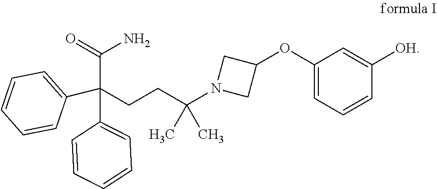 [SMILES: CC(C)(CCC(C(N)=O)(c1ccccc1)c1ccccc1)N1CC(Oc2cccc(O)c2)C1]

The term “dry powder inhalers” (DPI), refers to a device that delivers medication to the lungs in the form of a dry powder. DPIs are commonly used to treat respiratory diseases such as asthma, bronchitis, emphysema and COPD although DPIs. DPIs can be divided into two basic types: i) single dose inhalers, for the administration of a single dose of the active compound; and ii) multi-dose inhalers pre-loaded with quantities of active principles sufficient for longer treatment cycles.

The term “unit dose” “unit dose receptacle” and/or “dose unit” refers to a container, such as a pocket or receptacle that contains a medicament configured to be dispensed to a patient at a particular dose.

The term “container” refers to a receptacle for a medicament. In some embodiments, the container can have one or more pockets for the medicament.

The term “boundary layer interface” refers to an area between layers of a medicament. It will be understood by those of ordinary skill in the art that there is a small degree of mixing when a medicament is layered and a small degree of mixing between layers during transportation of the medicament.

The term “excipient” is used to describe an ingredient other than the active pharmaceutical ingredients. The selection of an excipient can depend on factors such as the particular mode of administration, the effect of the excipient on solubility and stability, and the nature of the dosage form.

The headings below are not meant to limit the disclosure in any way; embodiments under any one heading may be used in conjunction with embodiments under any other heading.

Reference will now be made in detail to certain embodiments of the invention, examples of which are illustrated in the accompanying drawings. While the invention will be described in conjunction with the illustrated embodiments, it will be understood that they are not intended to limit the invention to those embodiments.

### Medicaments

A medicament for inhalation is provided, the medicament comprising at least a first layer comprising a first active pharmaceutical ingredient (API) in powder form and at least a second layer comprising at least a second active pharmaceutical ingredient in powder form, the second layer disposed on the first layer. Therefore, the APIs are separated into different layers and are not substantially mixed or blended together before inhalation.

The medicament containing the APIs in layers should have the particle size and flowability to be administered by inhalation (e.g., oral or nasal inhalation). One preferred oral inhaler use for administration of the medicament is a dry powder inhaler. The API can be any API suitable for inhalation including an anticholinergic agent, a bronchodilator, and/or a corticosteroids.

The anticholinergic agents include, but are not limited to trihexyphenidyl, benztropine mesylate, ipratropium, tiotropium, orphenadrine, atropine, flavoxate, oxybutynin, scopolamine, hyoscyamine, tolterodine, belladonna alkaloids, fesoterodine, solifenacin, darifenacin, propantheline, 5-[3-(3-Hydroxyphenoxy)azetidin-1-yl]-5-methyl-2,2-diphenylhexanamide or a combination thereof. The bronchodilators include, but are not limited to, adrenergic bronchodilators including, but not limited to, levalbuterol, metaproterenol, pirbuterol, formoterol, terbutaline, or albuterol; anticholinergic bronchodilators including, but not limited to, aclidinium systemic, ipratropium systemic, tiotropium systemic, orumeclidinium systemic; bronchodilator combinations including, but not limited to, umeclidinium/vilanterol systemic, budesonide/formoterol systemic, fluticasone/salmeterol systemic, albuterol/ipratropium systemic, fluticasone/vilanterol systemic, olodaterol/tiotropium systemic, formoterol/mometasone systemic, formoterol/glycopyrrolate systemic, orglycopyrrolate/indacaterol systemic; and methylxanthines including, but not limited to, theophylline systemic, aminophylline systemic, or dyphylline systemic. The corticosteroids include, but are not limited to, beclomethasone, budesonide, flunisolide, fluticasone, mometasone, ciclesonide or tiotropium.

The API in the medicament stored may include the medicaments described above, and may also include substances traditionally regarded as actives, drugs and bioactive agents, as well as biopharmaceuticals (e.g., peptides, hormones, nucleic acids, gene constructs, etc.) typically employed to treat a number of conditions which is defined broadly to encompass diseases, disorders, infections, and the like. Exemplary APIs, without limitation, antibiotics, antivirals, H2-receptor antagonists, 5HT1 agonists, 5HT3 antagonists, COX2-inhibitors, APIs used in treating psychiatric conditions such as depression, anxiety, bipolar condition, tranquilizers, APIs used in treating metabolic conditions, anticancer APIs, APIs used in treating neurological conditions such as epilepsy and Parkinson's Disease, APIs used in treating cardiovascular conditions, non-steroidal anti-inflammatory APIs, APIs used in treating Central Nervous System conditions, and APIs employed in treating hepatitis.

In some embodiments, the APIs may also be selected from, analgesics, e.g., codeine, dihydromorphine, ergotamine, fentanyl or morphine; anginal preparations, e.g., diltiazem; antiallergics, e.g., cromoglycate (e.g., as the sodium salt), ketoprofen or nedocromil (e.g., as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g., as the dipropionate ester), fluticasone (e.g., as the propionate ester), flunisolide, prednisone, prednisolone, budesonide, rofleponide, mometasone e.g. as the furoate ester), ciclesonide, triamcinolone (e.g. as the acetonide) or 6α,9α-difluoro-11β-hydroxy-16α-methyl-3-oxo-17α-propionyloxy-androsta-1,4-diene-17β-carbothioic acid S-(2-oxo-tetrahydro-furan-3-yl)ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g., as fumarate), isoprenaline, metaproterenol, phenylephrine, phenylpropanolamine, pirbuterol (e.g., as acetate), reproterol (e.g., as hydrochloride), rimiterol, terbutaline (e.g., as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; adenosine 2a agonists, e.g., 2R,3R,4S,5R)-2-[6-Amino-2-(1S -hydroxymethyl-2-phenyl-ethylamino)purin-9-yl]-5-(2-ethyl-2H-tetrazol-5-yl)-tetrahydro-furan-3,4-diol (e.g. as maleate); α4 integrin inhibitors e.g. (2S)-3-[4-({[4-(aminocarbonyl)-1-piperidinyl]carbonyl}oxy)phenyl]-2-[((2S)-4-methyl-2-{[2-(2-methylphenoxy)acetyl]amino}pentanoyl)amino]propanoic acid (e.g., as free acid or potassium salt), diuretics, e.g., amiloride; anticholinergics, e.g., ipratropium (e.g., as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the APIs may be used in the form of salts, (e.g., as alkali metal or amine salts or as acid addition salts) or as esters (e.g., lower alkyl esters) or as solvates (e.g., hydrates) to optimize the activity and/or stability of the API.

Additionally, the APIs may be selected from, for example, antibiotics. Such antibiotics include, for example, nitroimidazole antibiotics, tetracyclines, penicillins, cephalosporins, carbopenems, aminoglycosides, macrolide antibiotics, lincosamide antibiotics, 4-quinolones, rifamycins and nitrofurantoin. In the following examples of such antibiotics are listed: ampicillin, amoxicillin, benzylpenicillin, phenoxymethylpenicillin, bacampicillin, pivampicillin, carbenicillin, cloxacillin, cyclacillin, dicloxacillin, methicillin, oxacillin, piperacillin, ticarcillin, flucloxacillin, cefuroxime, cefetamet, cefetrame, cefixine, cefoxitin, ceftazidime, ceftizoxime, latamoxef, cefoperazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldimycin, lincomycirl, vancomycin, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametoxazol, bismuth subsalicylate, colloidal bismuth subcitrate, gramicidin, mecillinam, cloxiquine, chlorhexidine, dichlorobenzylalcohol, methyl-2-pentylphenol. The active antibiotics could be in standard forms or used as salts, hydrates, esters etc. A combination of two or more of the above listed drugs may be used. The antibiotics can be clarithromycin, erythromycin, roxithromycin, azithromycin, amoxicillin, metronidazole, tinidazole and tetracycline. Clarithromycin and metronidazole alone or in combination are especially suitable.

Moreover, APIs may also be selected from, for example, antivirals. Examples of APIs that are effective for the treatment of viral and viral associated conditions are (1-alpha, 2-beta, 3-alpha)-9-[2,3-bis(hydroxymethyl)cyclobutyl]guanine [(−)BHCG, SQ-34514, lobucavir], 9-[(2R,3R,4S)-3,4-bis(hydroxymethyl)-2-oxetanosyl]adenine (oxetanocin-G), acyclic nucleosides, for example acyclovir, valaciclovir, famciclovir, ganciclovir, and penciclovir, acyclic nucleoside phosphonates, for example, (S)-1-(3-hydroxy-2-phosphonyl-methoxypropyl)cytosine (HPMPC), [[[2-(6-amino-9H-purin-9-yl)ethoxy]methyl]phosphinylidene]bis(oxymethylene)-2,2-dimethylpropanoic acid (bis-POM PMEA, adefovir dipivoxil), [[(1R)-2-(6-amino-9H-purin-9-yl)-1-methylethoxy]methyl]phosphonic acid(tenofovir), and (R)-[[2-(6-Amino-9H-purin-9-yl)-1-methylethoxy]methyl]phosphonic acid bis-(isopropoxy carbonyloxymethyl)ester (bis-POC-PMPA), ribonucleotide reductase inhibitors, for example, 2-acetylpyridine 5-[(2-chloroanilino)thiocarbonyl)thiocarbonohydrazone and hydroxyurea, nucleoside reverse transcriptase inhibitors, for example 3′-azido-3′-deoxythymidine (AZT, zidovudine), 2′,3′-dideoxycytidine (ddC, zalcitabine), 2′,3′-dideoxyadenosine, 2′,3′-dideoxyinosine (ddI, didanosine), 2′,3′-didehydrothymidine (d4T, stavudine), (−)-beta-D-2,6-diaminopurine dioxolane (DAPD), 3′-azido-2′,3′-dideoxythymidine-5′-H-phosphophonate (phosphonovir), 2′-deoxy-5-iodo-uridine (idoxuridine), (−)-cis-1-(2-hydroxymethyl)-1,3-oxathiolane 5-yl)-cytosine (lamivudine), cis-1-(2-(hydroxymethyl)-1,3-oxathiolan-5-yl)-5-fluorocytosine (FTC), 3′-deoxy-3′-fluorothymidine, 5-chloro-2′,3′-dideoxy-3′-fluorouridine, (−)-cis-4-[2-amino-6-(cyclopropylamino)-9H-purin-9-yl]-2-cyclopentene-1-methanol (abacavir), 9-[4-hydroxy-2-(hydroxymethyl)but-1-yl]-guanine (H2G), ABT-606 (2HM-H2G) and ribavirin, protease inhibitors, for example indinavir, ritonavir, nelfinavir, amprenavir, saquinavir, (R)-N-tert-butyl-3-[(2S,3S)-2-hydroxy-3-N-[(R)-2-N-(isoquinolin-5-yloxyacetyl)amino-3-methylthio-propanoyl]amino-4-phenylbutanoyl]-5,5-dimethyl-1,3-thiazolidine-4-carboxamide (KNI-272), 4R-(4alpha,5alpha,6beta)]-1,3-bis [(3-aminophenyl)methyl]hexahydro-5,6-dihydroxy-4,7-bis(phenylmethyl)-2H-1,3-diazepin-2-one dimethanesulfonate (mozenavir), 3-[1-[3-[2-(5-trifluoromethylpyridinyl)-sulfonylamino]phenyl]propyl]-4-hydroxy-6alpha-phenethyl-6beta-propyl-5,6-dihydro-2-pyranone (tipranavir), N′-[2(S)-Hydroxy-3(S)-[N-(methoxycarbonyl)-I-tert-leucylamino]-4-phenylbutyl-Nalpha-(methoxycarbonyl)-N′-[4-(2-pyridyl)benzyl]-L-tert-leucylhydrazide (BMS-232632), 3-(2(S)-Hydroxy-3(S)-(3-hydroxy-2-methylbenzamido)-4-phenylbutanoyl)-5,5-dimethyl-N-(2-methylbenzyl)thiazolidine-4(R)-carboxamide (AG-1776), N-(2(R)-hydroxy-1(S)-indanyl)-2(R)-phenyl-methyl-4(S)-hydroxy-5-(1-(1-(4-benzo[b]furanylmethyl)-2(S)-N′-(tert-butylcarboxamido)piperazinyl)pentanamide (MK-944A), and (3S)-tetrahydrofuran-3-yl (1S,2R)-[[(4-aminophenyl)sulphonyl)](isobutyl)amino]-1-benzyl-2-(phosphonooxy)propylcarbamate monocalcium salt (fosamprenavir), interferons such as α-interferon, renal excretion inhibitors such as probenecid, nucleoside transport inhibitors such as dipyridamole; pentoxifylline, N-acetylcysteine (NAC), Procysteine, α-trichosanthin, phosphonoformic acid, as well as immunomodulators such as interleukin II or thymosin, granulocyte macrophage colony stimulating factors, erythropoetin, soluble CD4 and genetically engineered derivatives thereof, non-nucleoside reverse transcriptase inhibitors (NNRTIs), for example nevirapine (BI-RG-587), alpha-((2-acetyl-5-methylphenyl)amino)-2,6-dichloro-benzeneacetamide (loviride), 1-[3-(isopropylamino)-2-pyridyl]4-[5-(methanesulfonamido)-1H-indol-2-ylcarbonyl]piperazine monomethanesulfonate (delavirdine), (10R,11S,12S)-12-Hydroxy-6,6,10,11-tetramethyl-4-propyl-11,12-dihydro-2H,6H,10H-benzo(1,2-b:3,4-b′:5,6-b″)tripyran-2-one ((+) calanolide A), (4S)-6-Chloro-4-[1E)-cyclopropylethenyl)-3,4-dihydro-4-(trifluoromethyl)-2(1H)-quinazolinone (DPC-083), (S)-6-chloro-4-(cyclopropylethynyl)-1,4-dihydro4-(trifluoromethyl)-2H-3,1-benzoxazin-2-one (efavirenz, DMP 266), 1-(ethoxymethyl)-5-(1-methylethyl)-6-(phenylmethyl)-2,4(1H,3H)-pyrimidinedione (MKC-442), and 5-(3,5-dichlorophenyl)thio-4-isopropyl-1-(4-pyridyl)methyl-1H-imidazol-2-ylmethyl carbamate (capravirine), glycoprotein 120 antagonists, for example, PRO-2000, PRO-542 and 1,4-bis[3-[(2,4-dichlorophenyl)carbonylamino]-2-oxo-5,8-disodiumsulfanyl]naphthalyl-2,5-dimethoxyphenyl-1,4-dihydrazone (FP-21399), cytokine antagonists, for example reticulose (Product-R), 1,1′-azobis-formamide (ADA), 1,11-(1,4-phenylenebis(methylene))bis-1,4,8,11-tetraazacyclotetradecane octahydrochloride (AMD-3100), integrase inhibitors, for example, S-1360, and fusion inhibitors.

The medicament may also include pharmaceutically acceptable salts, esters, solvates, and/or hydrates of the pharmaceutically active substances referred to hereinabove. Various combinations of any of the above medicaments may also be employed.

In some embodiments, an inhalation powder medicament is provided that has a multi-layered filling to create a combination layered dry powder medicament. In some embodiments, the layered dry powder medicament can be made of a layer of an anticholinergic agent in the first layer and a layer of a bronchodilator in the second layer. In some embodiments, the layered dry powder medicament can be made of a layer of an anticholinergic agent in the first layer and a layer of a corticosteroid in the second layer. In some embodiments, the layered dry powder medicament can be made of a layer of a bronchodilator in the first layer and a layer of a corticosteroid in the second layer.

In some embodiments, the layered dry powder medicament can be made of a layer of an anticholinergic agent in the first layer, a layer of a bronchodilator in the second layer and a layer of a corticosteroid in the third layer.

In some embodiments, the layered dry powder medicament can be made of a layer of an anticholinergic agent comprising 5-[3-(3-Hydroxyphenoxy)azetidin-1-yl]-5-methyl-2,2-diphenylhexanamide hydrochloride or 5-[3-(3-Hydroxyphenoxy)azetidin-1-yl]-5-methyl-2,2-diphenylhexanamide in a first layer, a bronchodilator comprising salmeterol xinafoate in a second layer and a corticosteroid comprising fluticasone propionate in a third layer. In some embodiments, the layered dry powder medicament can be made of a layer of an anticholinergic agent comprising 5-[3-(3-Hydroxyphenoxy)azetidin-1-yl]-5-methyl-2,2-diphenylhexanamide hydrochloride or 5-[3-(3-Hydroxyphenoxy)azetidin-1-yl]-5-methyl-2,2-diphenylhexanamide in a first layer and a bronchodilator comprising salmeterol xinafoate or a corticosteroid comprising fluticasone propionate in a second layer. In some embodiments, the layered dry powder medicament can be made of a layer of a bronchodilator comprising salmeterol xinafoate and a corticosteroid comprising fluticasone propionate in a second layer.

In some embodiments, an inhalation powder medicament is provided that has a multi-layered filling to create a combination layered dry powder medicament. In some embodiments, the layered dry powder medicament can be made of a layer of an anticholinergic agent comprising 5-[3-(3-Hydroxyphenoxy)azetidin-1-yl]-5-methyl-2,2-diphenylhexanamide hydrochloride or 5-[3-(3-Hydroxyphenoxy)azetidin-1-yl]-5-methyl-2,2-diphenylhexanamide combined with a layer of a bronchodilator comprising salmeterol xinafoate and a corticosteroid comprising fluticasone propionate. 5-[3-(3-Hydroxyphenoxy)azetidin-1-yl]-5-methyl-2,2-diphenylhexanamide hydrochloride or its free base 5-[3-(3-Hydroxyphenoxy)azetidin-1-yl]-5-methyl-2,2-diphenylhexanamide is a long acting anticholinergic that can be used in the dose range of about 40 to about 800 mcg in 7 mg of blend, or a dose range of about 300 to about 600 mcg.

The layered dry powder medicament can be configured for disposal in an inhaler device, such as the device disclosed in U.S. Pat. No. 9,399,103. The entire disclosure is herein incorporated by reference.

In some embodiments, a bi-layered dry powder medicament can be made from a long-acting anticholinergic, long-acting beta-agonist and inhaled corticosteroid (LAAC/LABA/ICS) combination therapy of a 12.5 mg first layer of 50 mcg salmeterol (as 72.5 mcg salmeterol xinafoate salt) with either 100, 250 or 500 mcg fluticasone propionate in 12.5 mg of blend and an excipient, and a 7 mg second layer of 5-[3-(3-Hydroxyphenoxy)azetidin-1-yl]-5-methyl-2,2-diphenylhexanamide hydrochloride and an excipient. In some embodiments, the dry powder product of the layered dry powder medicament has a layering fill weight with a 7 mg layer of 5-[3-(3-Hydroxyphenoxy)azetidin-1-yl]-5-methyl-2,2-diphenylhexanamide hydrochloride and an excipient, followed by 13 mg of salmeterol xinafoate, fluticasone propionate and an excipient.

The layered dry powder medicament may be applicable to any combination of orally inhaled drug combinations. In some embodiments, the layered dry powder medicament is for asthma or COPD treatment which can be layered or multi-dosed into a unit dose receptacle such as a capsule or blister. The pocket of a dose carrier with pockets in the device of U.S. Pat. No. 9,399,103 may be used. In a bi-layered configuration, two layers can be provided having a 7-13 mg or 13-7 mg fill weight layer combination in the pockets of a dose carrier with pockets. In some embodiments, two layers can be provided having a fill weight ratio of 10:10 or 2.5:17.5 layer combination in the pockets of a dose carrier with pockets. In some embodiments, a three layer configuration may also be provided having a 7-7-7 mg fill weight and the layers can be made via filling heads.

In some embodiments, the layered dry powder medicament can have multiple layers made of any combination drug blends and can be layered with re-designed fillings heads. Nominal dose carrier pocket volume can be 31 μl, and the usable pocket volume can be up to 26 μl with a minimum available pocket volume of the first layer being 9.1 μl. The inhalation blend(s) can be filled with a tapped density in the range of about 0.6 to about 0.9 mg/μl, or about 0.7 to about 0.8 mg/μl. In some embodiments, a multiple number of layers can be filled with different fill weights from a minimum first layer fill weight of 2.5 mg up to a total fill weight of the multi-dose of up to 23 mg. The actual fill weight achievable will be dependent on the physical characteristics of the powders filled.

The layered dry powder medicament can apply to any orally inhaled drugs that can be given as a combination dry powder therapy in the dry powder form. The layered dry powder medicament can be applied to any filling technology that fills powder into a unit dose receptacle, either in a layering or multi-dose fill process. In some embodiments, the filling technology pre-meters the dose prior to filling into the unit dose (dosator-like technology), or is capable of partially filling the dose into the unit dose (e.g., the filling technology disclosed in U.S. Pat. No. 9,399,103).

For example, capsule filling technology may use a dosator to put a predefined weight of powder into a capsule without completely filling the capsule. The available volume after the initial first fill could be used to fill a second powder using a second dosator turret. Omnidose® (Harro Höfliger) may also be another filling technology that could be capable of double filling a receptacle by having two dosating drums in series. In some embodiments, Xcelodose® (Capsugel) filling technology may also be used.

The present application provides a medicament for inhalation that is multi-layered and maintains the medicament's performance characteristics prior to administration to a patient. The medicament is in dry powder form and is configured for use in a dry powder inhaler (DPI), such as the DPI device disclosed in U.S. Pat. No. 9,399,103, the contents of which are incorporated herein by reference. The medicament may also be compatible with other unit dose or multi dose delivery devices. The medicament is for either oral or nasal inhalation.

The medicament comprises at least a first layer comprising a first active pharmaceutical ingredient (API) in powder form, and at least a second layer comprising at least a second API in powder form. The first API and the at least second API are not the same API. The second layer of the medicament is disposed on the first layer. In some embodiments, the first layer may be disposed on the second layer. The first layer and the second layer comprise micronized powder. In some embodiments, the API may be in a crystalline, amorphous form or a combination thereof.

In some embodiments, the first API of the at least first layer comprises an anticholinergic agent, and the at least second API of the at least second layer comprises both a bronchodilator and a corticosteroid.

In some embodiments, the anticholinergic agent comprises 5-[3-(3-Hydroxyphenoxy)azetidin-1-yl]-5-methyl-2,2-diphenylhexanamide hydrochloride having the structure of

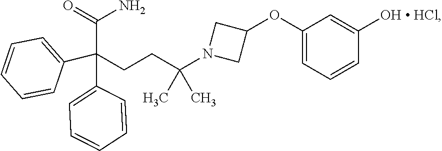

where the bronchodilator can comprise optionally salmeterol xinafoate and/or optionally the corticosteroid comprises fluticasone propionate.

In some embodiments, the anticholinergic agent comprises 5-[3-(3-Hydroxyphenoxy)azetidin-1-yl]-5-methyl-2,2-diphenylhexanamide having the structure of

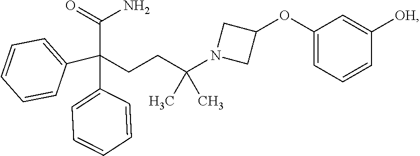 [SMILES: CC(C)(CCC(C(N)=O)(c1ccccc1)c1ccccc1)N1CC(Oc2cccc(O)c2)C1]

where the bronchodilator can comprise optionally salmeterol xinafoate and/or optionally the corticosteroid comprises fluticasone propionate.

In some embodiments, the 5-[3-(3-Hydroxyphenoxy)azetidin-1-yl]-5-methyl-2,2-diphenylhexanamide hydrochloride is in a dose of about 40 to about 800 mcg, the salmeterol xinafoate is in a dose of about 72.5 mcg, and the fluticasone propionate is in a dose of about 100 to about 500 mcg.

The 5-[3-(3-Hydroxyphenoxy)azetidin-1-yl]-5-methyl-2,2-diphenylhexanamide hydrochloride is in a dose of from about 40 to about 800 mcg. In some embodiments, the 5-[3-(3-Hydroxyphenoxy)azetidin-1-yl]-5-methyl-2,2-diphenylhexanamide hydrochloride is in a dose of from about 40 to about 700 mcg, from about 40 to about 600 mcg, from about 40 to about 500 mcg, from about 40 to about 400 mcg, from about 40 to about 300 mcg, from about 40 to about 200 mcg, from about 40 to about 100 mcg, from about 100 to about 800 mcg, from about 100 to about 700 mcg, from about 100 to about 600 mcg, from about 100 to about 500 mcg, from about 100 to about 400 mcg, from about 100 to about 300 mcg, from about 100 to about 200 mcg, or from about 100 to about 150 mcg.

In some embodiments, the 5-[3-(3-Hydroxyphenoxy)azetidin-1-yl]-5-methyl-2,2-diphenylhexanamide hydrochloride is in a dose of from about 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690 to about 700 mcg.

In some embodiments, the 5-[3-(3-Hydroxyphenoxy)azetidin-1-yl]-5-methyl-2,2-diphenylhexanamide hydrochloride is in an amount from about 0.01 to about 99 wt. % of the medicament and/or the at least first layer. In some embodiments, the -[3-(3-Hydroxyphenoxy)azetidin-1-yl]-5-methyl-2,2-diphenylhexanamide hydrochloride is in an amount from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15,16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 85, 86, 87, 88, 89, 90, 91, 92, 93, 94, 95, 96, 97, 98 to about 99 wt % of the medicament and/or the at least first layer.

The salmeterol xinafoate is in a dose of about 72.5 mcg. In some embodiments, the salmeterol xinafoate is in a dose of from about 5 to about 100 mcg, from about 5 to about 75 mcg, from about 10 to about 100 mcg, from about 10 to about 75 mcg, from about 10 to about 50 mcg, from about 25 to about 100 mcg, from about 25 to about 75 mcg, or from about 25 to about 50 mcg.

In some embodiments, the salmeterol xinafoate is in a dose from about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100 mcg.

In some embodiments, the salmeterol xinafoate is in an amount of about 0.01 to about 99 wt. % of the medicament and/or the second layer. In some embodiments, the salmeterol xinafoate is in an amount from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 85, 86, 87, 88, 89, 90, 91, 92, 93, 94, 95, 96, 97, 98 to about 99 wt % of the medicament and/or the second layer.

In some embodiments, the fluticasone propionate is in a dose of about 100 to about 500 mcg. In some embodiments, the fluticasone propionate is in a dose of about 100 to about 400 mcg, about 100 to about 300 mcg, about 100 to about 200 mcg, about 100 to about 150 mcg, about 200 to about 500 mcg, about 200 to about 400 mcg, about 200 to about 300 mcg, about 300 to about 500 mcg, or about 300 to about 400 mcg.

In some embodiments, the fluticasone propionate is in a dose of from about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 to about 500 mcg.

In some embodiments, the fluticasone propionate is in an amount of from about 0.01 to about 99 wt. % of the medicament and/or the second layer. In some embodiments, the fluticasone propionate is in an amount from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15,16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 85, 86, 87, 88, 89, 90, 91, 92, 93, 94, 95, 96, 97, 98 or 99 wt % of the medicament and/or the second layer.

In some embodiments, the medicament including the API and excipient have a mean particle size particle size of from about 1 μm to about 500 μm.

In some embodiments, the fluticasone propionate has a particle size from about less than 10 μm to about less than 3 μm. In some embodiments, the fluticasone propionate has a particle size of from about less than 3 μm, less than 4 μm, less than 5 μm, less than 6 μm, less than 7 μm, less than 8 μm, less than 9 μm, to about less than 10 μm. In some embodiments, the fluticasone propionate has a D50 value of about 1 to about 3 μm. In some embodiments, the fluticasone propionate has a D90 value of about 4 to about 6 μm.

In some embodiments, the 5-[3-(3-Hydroxyphenoxy)azetidin-1-yl]-5-methyl-2,2-diphenylhexanamide hydrochloride has a particle size of from about less than 10 μm to about less than 1 μm. In some embodiments, the 5-[3-(3-Hydroxyphenoxy)azetidin-1-yl]-5-methyl-2,2-diphenylhexanamide hydrochloride has a particle size of from about less than 1 μm, less than 2 μm, less than 3 μm, less than 4 μm, less than 5 μm, less than 6 μm, less than 7 μm, less than 8 μm, less than 9 μm, to about less than 10 μm.

In some embodiments, the salmeterol xinafoate has a particle size of from about less than 10 μm to about less than 1 μm. In some embodiments, the salmeterol xinafoate has a particle size of from about less than 1 μm, less than 2 μm, less than 3 μm, less than 4 μm, less than 5 μm, less than 6 μm, less than 7 μm, less than 8 μm, less than 9 μm, to about less than 10 μm.

The at least first layer of the medicament further comprises a first excipient blended with the first API to form a first dry powder, and the at least second layer of the medicament further comprises a second excipient blended with the second API to form a second dry powder. The first and second excipients may be the same or different and can comprise glucose, arabinose, lactose, sucrose, maltose, mannitol, dextrans, magnesium stearate, leucine, isoleucine, lysine, valine, methionine, phenylalanine, or a combination thereof. In some embodiments, the lactose is lactose monohydrate. In some embodiments, the excipient can include, but is not limited to, monosaccharides such as galactose, mannose, sorbose; disaccharides such as lactose, sucrose and trehalose and the like; polysaccharides such as starch, raffinose, dextran and the like; and mixtures thereof. In some embodiments, the at least first layer and/or the at least second layer can comprise more than one excipient.

In some embodiments, the at least first layer and the at least second layer comprise an excipient comprising lactose monohydrate, and the lactose monohydrate can be coarse, fine or a mixture thereof. In some embodiments, the lactose monohydrate is Lactohale 200 (LH200) or Lactohale 230 (LH230). The LH200 can have a particle size of D₁₀ of from about 5 to about 15 μm, a particle size of D₅₀ of from about 50 μm to about 100 μm, and a particle size of D₉₀ of from about 120 to about 160 μm. The LH230 can have a particle size of D₁₀ of from about 1 to about 3 μm, a particle size of D₅₀ of greater than 10 μm, and a particle size of D₉₀ of greater than 30 μm.

In some embodiments, the excipient comprises from about 0.01 to about 99.9 wt. % of the first layer, the second layer or the entire medicament. In some embodiments, the excipient comprises from about 0.1 to about 99 wt. %, from about 1 to about 99 wt. %, from about 10 to about 99 wt. %, from about 20 to about 99 wt. %, from about 30 to about 99 wt. %, from about 40 to about 99 wt. %, from about 50 to about 99 wt. %, from about 60 to about 99 wt. %, from about 70 to about 99 wt. %, from about 80 to about 99 wt. %, from about 90 to about 99 wt. %, from about 95 to about 99 wt. %, or from about 97 to about 99 wt. % of the first layer, the second layer or the entire medicament.

In some embodiments, the excipient comprises from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 99.9 wt. % of the first layer, the second layer and/or the entire medicament.

In some embodiments, the excipient is a powder and has an average particle size of from about 10 to about 200 μm. In some embodiments, the excipient has an average particle size of from about 10 to about 150 μm, from about 10 to about 100 μm, from about 10 to about 75 μm, from about 10 to about 50 μm, from about 25 to about from about 150 μm, from about 25 to about 100 μm, from about 25 to about 75 μm, or from about 25 to about 50 μm. In some embodiments, the excipient has an average particle size of from about 10, 20, 30, 40, 50, 60, 70, 80, 90, 100, 110, 120, 130, 140, 150, 160, 170, 180, 190 to about 200 μm. The excipient can be, in some embodiments, lactose.

In some embodiments, the first layer comprises the first API 5-[3-(3-Hydroxyphenoxy)azetidin-1-yl]-5-methyl-2,2-diphenylhexanamide hydrochloride and the first excipient is in an amount of from about 3 to about 9 mg, and the second layer comprises the at least second API salmeterol xinafoate combined with fluticasone propionate and the second excipient is in an amount of from about 8 to about 13 mg.

In some embodiments, the first layer comprising the first API and the first excipient can be in an amount of from about 3, 4, 5, 6, 7, 8 or 9 mg, and the second layer comprising the at least second API and the second excipient can be in an amount of from about 9, 10, 11, 12 or 13 mg.

In some embodiments, the second layer of the medicament is disposed on the first layer in a linear configuration. In some embodiments, the second layer is disposed on the first layer in a non-linear configuration. In some embodiments, the first layer and/or the second layer are configured in various patterns such as, for example, in regular or irregular patterns. In some embodiments, a portion or the entire first layer and/or second layer is wavy, angled, arcuate, concave, convex, inclined, and/or declined. In some embodiments, a portion or the entire first layer and/or second layer is squared, circular, triangular, dimpled, toothed, inverted, flat, pentagonal, and/or hexagonal. In some embodiments, the first layer and/or the second layer are configured in various patterns via different filling head designs.

The at least first layer of the medicament and the at least second layer of the medicament do not intentionally mix together, however, in some embodiments, a boundary layer interface between the at least first layer and the at least second layer comprises a mixed layer comprising the APIs of the at least two layers. In some embodiments, the at least first layer and the at least second layer mix only at a boundary layer interface in an amount from about 0.01 to about 10 wt. %. In some embodiments, the at least first layer and the at least second layer mix only at a boundary layer interface in an amount from about 0.01 to about 10 wt. %, from about 0.1 to about 10 wt. %, from about 1 to about 10 wt. %, from about 1 to about 5 wt. %, or from about 5 to about 10 wt. %. In some embodiments, the at least first layer and the at least second layer mix only at a boundary layer interface in an amount from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9 to about 10 wt. %.

In some embodiments, the medicament can be bi-layered or tri-layered. In some embodiments, the medicament can have at least 1 to about 10 layers, at least 1 to about 8 layers, at least 1 to about 6 layers, at least 1 to about 4 layers, at least 1 to about 3 layers, or at least 1 to about 2 layers. In some embodiments, the medicament can have at least 1 to about 2, 3, 4, 5, 6, 7, 8, 9 or about 10 layers.

In some embodiments, the medicament has a density of from about 0.6 to about 0.9 mg/μl. In some embodiments, the medicament has a density of from about 0.6 to about 0.8 mg/μl, or from about 0.6 to about 0.7 mg/μl. In some embodiments, the medicament has a density of from about 0.6, 0.7, 0.8 or 0.9 mg/μl.

In some embodiments, the medicament is administered to the patient two times a day for a selected period of time. In some embodiments, the medicament is administered to the patient 1, 2, 3, or 4 times a day at a selected period of time. Each dose of medicament is administered between about 1 to about 12 hours after administration of the last dose. In some embodiments, each dose of the medicament is administered between about 1, 2, 3, 4, 5, 6, 7, 8, 9, 10, 11, or 12 hours after administration of the last dose.

In some embodiments, the at least first layer and/or the at least second layer of the medicament does not comprise a coating designed for sustained release in the gastrointestinal tract (e.g., enteric coatings). Sustained release coatings designed for the gastrointestinal tract that the medicament does not use, include, but are not limited to, cellulose acetate phthalate (CAP), hydroxypropyl methylcellulose phthalate (HPMCP), polyvinyl acetate phthalate (PVAP), hydroxypropylmethyl cellulose, hydroxypropyl methylcellulose acetate succinate (HPMCAS), cellulose acetate trimellitate, hydroxypropyl methylcellulose succinate, carboxymethyl cellulose, carboxymethyl ethyl cellulose, cellulose acetate phthalate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, polyvinyl acetate phthalate, zein, shellac, copal collophirium, Eduragit L30D55, Eudragit FS30D, Eudragit L100, Eudragit S100, Kollicoat EMM30D, Estacryl 30D, Coateric, Aquateric, or a combinations thereof.

In some embodiments, the medicament comprises, consists, or consists essentially of a first layer including an API and an excipient, and a second layer including an API/APIs and an excipient.

In some embodiments, the anticholinergic agents, bronchodilators and/or corticosteroids are in the at least first layer, the at least second layer and/or the medicament in an amount from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15,16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 85, 86, 87, 88, 89, 90, 91, 92, 93, 94, 95, 96, 97, 98 to about 99 wt % of the medicament.

In some embodiments, the anticholinergic agents, bronchodilators and/or corticosteroids have particle sizes from about less than 10 μm to about less than 5 μm. In some embodiments, the fluticasone propionate has a particle size of from about less than 5 μm, less than 6 μm, less than 7 μm, less than 8 μm, less than 9 μm, to about less than 10 μm.

In some embodiments, the medicament is compatible for use with inhalation delivery devices such as, for example, nebulizers, pMDIs, and DPIs.

In some embodiments, the medicament further comprises propellants, including, but not limited to, hydrofluoroalkane (HFA), such as chlorodifluoromethan, trifluoromonofluoroethane, chlorodifluoroethane, difluoroethane, heptafluoropropane or a combination thereof.

### Medicament Container

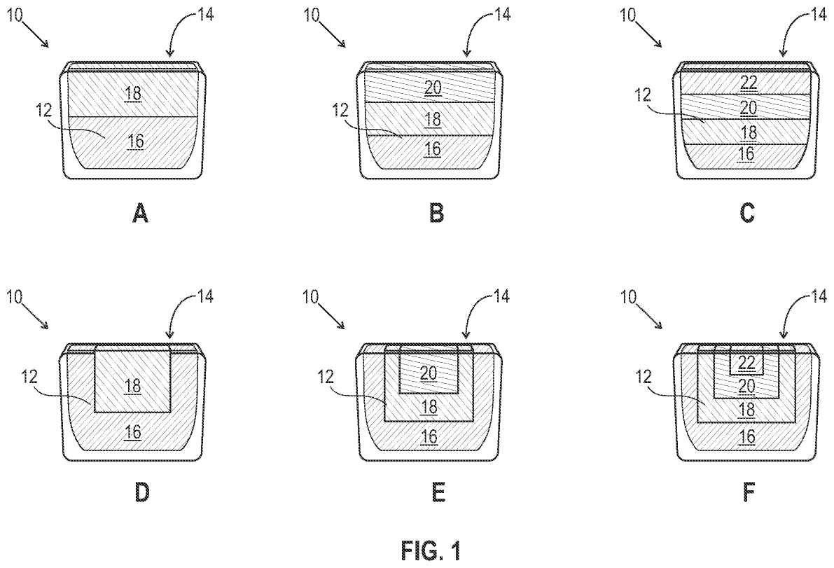

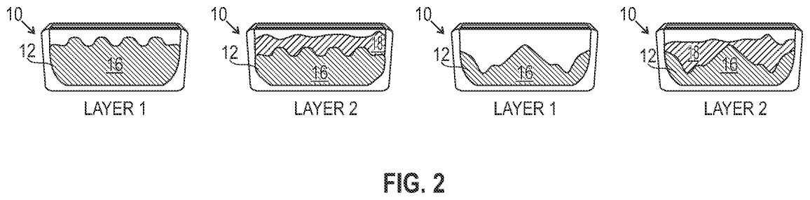

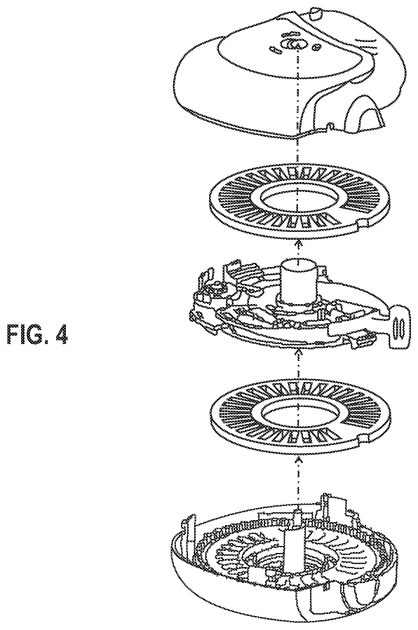

The present application provides a container 10 for dispensing of a medicament 12 for inhalation, as shown in FIGS. 1 and 2. The medicament container is compatible with the DPI device, as shown in FIG. 4, which is disclosed in U.S. Pat. No. 9,399,103. The medicament container is similar to the medicament containers disclosed in U.S. Pat. No. 9,399,103. The container may also be used in other inhalation delivery devices such as, for example, nebulizers, pMDIs, and other DPIs.

The container comprises an inner surface defining a pocket 14 configured for disposal and storage of the medicament, and a covering disposed over at least the pocket configured for piercing engagement such that the medicament can be dispensed from the container. The container dispenses the medicament orally or nasally. In one embodiment, the container is configured for administration via a dry powder inhaler device as shown in FIG. 4.

The medicament comprises at least a first layer 16 comprising a first API in powder form, and at least a second layer 18 comprising at least a second API in powder form, as described above with regard to the medicament. The first layer is disposed at a bottom of the pocket of the container, as shown in FIGS. 1A-F and 2. The first API and the at least second API are not the same API. The second layer is disposed on the first layer. In some embodiments, the first layer is disposed on the second layer.

As described above with regard to the medicament, the first API of the at least first layer, in some embodiments, comprises an anticholinergic agent comprising 5-[3-(3-Hydroxyphenoxy)azetidin-1-yl]-5-methyl-2,2-diphenylhexanamide hydrochloride, and the at least second API of the at least second layer comprises a bronchodilator comprising salmeterol xinafoate and/or a corticosteroid comprising fluticasone propionate.

In some embodiments, the 5-[3-(3-Hydroxyphenoxy)azetidin-1-yl]-5-methyl-2,2-diphenylhexanamide hydrochloride is in a dose of about 40 to about 800 mcg, the salmeterol xinafoate is in a dose of about 72.5 mcg, and the fluticasone propionate is in a dose of about 100 to about 500 mcg, as described above.

The at least first layer of the medicament further comprises a first excipient blended with the first API to form a first dry powder, and the at least second layer of the medicament further comprises a second excipient blended with the second API to form a second dry powder. The first and second excipients may be the same or different and can comprise an excipient comprising glucose, arabinose, lactose, sucrose, maltose, dextrans, magnesium stearate or a combination thereof. In some embodiments, the excipient can be lactose monohydrate. In some embodiments, the excipient can include, but is not limited to, monosaccharides such as galactose, mannose, sorbose; disaccharides such as lactose, sucrose and trehalose and the like; polysaccharides such as starch, raffinose, dextran and the like; and mixtures thereof. In some embodiments, the at least first layer and/or the at least second layer can comprise more than one excipient.

In some embodiments, the at least first layer and the at least second layer comprise an excipient comprising lactose monohydrate. In some embodiments, the lactose monohydrate is Lactohale 200 (LH200) or Lactohale 230 (LH230). The LH200 can have a particle size of D₁₀ of from about 5 to about 15 μm, a particle size of D₅₀ of from about 50 μm to about 100 μm, and a particle size of D₉₀ of from about 120 to about 160 μm. The LH230 can have a particle size of D₁₀ of from about 1 to about 3 μm, a particle size of D₅₀ of greater than 10 μm, and a particle size of D₉₀ of greater than 30 μm.

In some embodiments, the excipient comprises from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 99.9 wt. % of the first layer, the second layer and/or the entire medicament, as described above.

The first layer comprising the first API 5-[3-(3-Hydroxyphenoxy)azetidin-1-yl]-5-methyl-2,2-diphenylhexanamide hydrochloride and the first excipient is in an amount of from about 3 to about 9 mg, and the second layer comprising the second API salmeterol xinafoate combined with fluticasone propionate and the second excipient is in an amount of from about 8 to about 13 mg.

In some embodiments, the first layer comprising the first API and the first excipient can be in an amount of from about 3, 4, 5, 6 or 7 mg, and the second layer comprising the at least second API and the second excipient can be in an amount of from about 9, 10, 11, 12 or 13 mg. In some embodiments, the first layer and the second layer can be of equal weight each in an amount of from about 3, 4, 5, 6, 7, 8, 9, 10, 11, 12 or 13 mg.

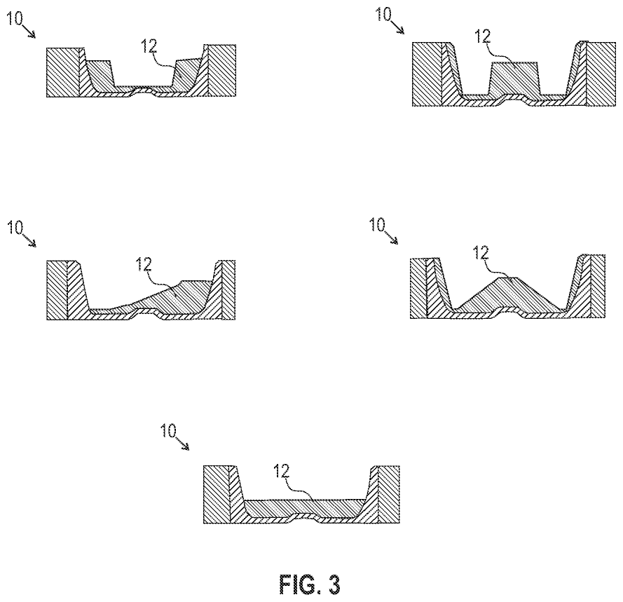

In some embodiments, the second layer of the medicament is disposed on the first layer in a linear configuration. In some embodiments, the second layer is disposed on the first layer in a non-linear configuration. In some embodiments, the first layer and/or the second layer are configured in various patterns such as, for example, in regular or irregular patterns, as shown in FIGS. 1-3. In some embodiments, a portion or the entire first layer and/or second layer is wavy, angled, arcuate, concave, convex, inclined, and/or declined. In some embodiments, a portion or the entire first layer and/or second layer is squared, circular, triangular, dimpled, toothed, inverted, flat, pentagonal, and/or hexagonal. In some embodiments, the first layer and/or the second layer are configured in various patterns via different filling head designs.

The at least first layer of the medicament and the at least second layer of the medicament do not intentionally mix together, however, in some embodiments, a boundary layer interface between the at least first layer and the at least second layer comprises a mixed layer comprising the APIS of the at least two layers. In some embodiments, the at least first layer and the at least second layer mix only at a boundary layer interface in an amount from about 0.01 to about 10 wt. %. In some embodiments, the at least first layer and the at least second layer mix only at a boundary layer interface in an amount from about 0.01 to about 10 wt. %, from about 0.1 to about 10 wt. %, from about 1 to about 10 wt. %, from about 1 to about 5 wt. %, or from about 5 to about 10 wt. %. In some embodiments, the at least first layer and the at least second layer mix only at a boundary layer interface in an amount from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9 to about 10 wt. %.

In some embodiments, the medicament can be bi-layered, tri-layered or have 4 layers, as shown in FIG. 1 in containers A, D, B, E, C, and F respectively. In some embodiments, the first layer, second layer, third layer, and/or fourth layer each comprise one or more APIs and an excipient. In the embodiment shown, the first layer 16, second layer 18, third layer 20, and/or fourth layer 22, each comprise one API and excipient. Containers A-C illustrate linear layer configurations and containers D-F illustrate non-linear layer configurations.

In some embodiments, the medicament can have at least 1 to about 10 layers, at least 1 to about 8 layers, at least 1 to about 6 layers, at least 1 to about 4 layers, at least 1 to about 3 layers, or at least 1 to about 2 layers. In some embodiments, the medicament can have at least 1 to about 2, 3, 4, 5, 6, 7, 8, 9, or 10 layers. The layers may each comprise at least one API and an excipient.

In some embodiments, the medicament comprises a first layer comprising 7 mg of 5-[3-(3-Hydroxyphenoxy)azetidin-1-yl]-5-methyl-2,2-diphenylhexanamide hydrochloride or -(3-Hydroxyphenoxy)azetidin-1-yl]-5-methyl-2,2-diphenylhexanamide and an excipient, a second layer comprising 7 mg of salmeterol xinafoate and an excipient, and a third layer comprising 7 mg fluticasone propionate and an excipient.

In some embodiments, the at least first layer and/or the at least second layer of the medicament does not comprise a coating designed for sustained release in the gastrointestinal tract (e.g., enteric coatings). Sustained release coatings designed for the gastrointestinal tract that the medicament does not use, include, but are not limited to, cellulose acetate phthalate (CAP), hydroxypropyl methylcellulose phthalate (HPMCP), polyvinyl acetate phthalate (PVAP), hydroxypropylmethyl cellulose, hydroxypropyl methylcellulose acetate succinate (HPMCAS), cellulose acetate trimellitate, hydroxypropyl methylcellulose succinate, carboxymethyl cellulose, carboxymethyl ethyl cellulose, cellulose acetate phthalate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, polyvinyl acetate phthalate, zein, shellac, copal collophirium, Eduragit L30D55, Eudragit FS30D, Eudragit L100, Eudragit S100, Kollicoat EMM30D, Estacryl 30D, Coateric, Aquateric, or a combinations thereof.

In some embodiments, the medicament comprises, consists, or consists essentially of a first layer including an API and an excipient, and a second layer including an API/APIs and an excipient.

In some embodiments, the medicament has a density of from about 0.6 to about 0.9 mg/μl within the pocket of the container. In some embodiments, the medicament has a density of from about 0.6 to about 0.8 mg/μl within the pocket of the container, or from about 0.6 to about 0.7 mg/μl. In some embodiments, the medicament has a density of from about 0.6, 0.7, 0.8 or 0.9 mg/μl within the pocket of the container.

The pocket of the container has a pocket volume that is from about 25 to about 32 μl. In some embodiments, the pocket of the container can have a pocket volume of about 25 to about 31 μl, from about 26 to about 30 μl, from about 27 to about 29 μl or from about 28 to about 30 μl. In some embodiments, the pocket volume is from about 25, 26, 27, 28, 29, 30, 31 or 32 μl. In some embodiments, the pocket volume is less than 31 μl. In some embodiments, the medicament utilizes about 26 μl of the pocket volume. In some embodiments, the medicament utilizes from about 10, 11, 12, 13, 14, 15, 16, 17, 18, 19, 20, 21, 22, 23, 24, 25 to about 26 μl of the pocket volume of the container.

In some embodiments, the at least first layer and/or the at least second layer of the medicament utilizes from about 1 to about 26 μl of the pocket volume of the container. In some embodiments, the at least first layer and/or the at least second layer of the medicament utilizes from about 1, 2, 3, 4, 5, 6, 7, 8, 9, 10, 11, 12, 13, 14, 15, 16, 17, 18, 19, 20, 21, 22, 23, 24, 25 or 26 μl of the pocket volume of the container.

In some embodiments, the at least first layer comprises about 7 to about 9 μl of the first medicament and the first excipient and the second layer comprises about 12 to about 17 μl of the at least second medicament and the second medicament.

In some embodiments, the at least first layer and/or the at least second layer can occupy from about 1 to about 100% of the volume of the container. In some embodiments, the at least first layer and/or the at least second layer can occupy from about 1 to about 90%, from about 1 to about 75%, from about 1 to about 50%, from about 1 to about 25%, from about 1 to about 10%, from about 10 to about 90%, from about 10 to about 75%, from about 10 to about 50%, from about 10 to about 25%, from about 25 to about 90%, from about 25 to about 75%, from about 25 to about 50%, from about 50 to about 90%, from about 50 to about 75%, or from about 75 to about 90% of the volume of the container. In some embodiments, the at least first layer and/or the at least second layer can occupy from about 1, 5, 10, 15, 20, 25, 30, 35, 40, 45, 50, 55, 60, 65, 70, 75, 80, 85, 90, 95, or about 100% of the volume of the container.

The pocket of the container comprises a fill weight, and in some embodiments, the first API and the first excipient has a fill weight in an amount of from about 3 to about 9 mg, and the at least second API and the second excipient has a fill weight in an amount of from about 8 to about 13 mg. In some embodiments, the first API combined with the first excipient and/or the at least second API combined with the second excipient has a fill weight of from about 1, 2, 3, 4, 5, 6, 7, 8, 9, 10, 11, 12, 13, 14, 15, 16, 17, 18, 19 to about 20 mg.

In some embodiments, the medicament is compatible for use with inhalation delivery devices such as, for example, nebulizers, pMDIs, and DPIs. In some embodiments, the container can include a pocket that is compatible for use with inhalation delivery devices such as, for example, DPIs.

In some embodiments, the inhaler does not require a propellant particularly in DPIs. However, in some embodiments, the inhaler can contain propellants, including, but not limited to, hydrofluoroalkane (HFA), such as chlorodifluoromethan, trifluoromonofluoroethane, chlorodifluoroethane, difluoroethane, heptafluoropropane or a combination thereof.

### Methods of Making

The present disclosure also provides methods of making a medicament for inhalation. The method comprises: providing a container comprising a pocket; and disposing the medicament into the pocket of the container, wherein the medicament comprises at least a first layer comprising a first API in powder form and at least a second layer comprising at least a second API in powder form, the first layer disposed on the second layer, wherein the first layer is disposed on the second layer in a linear or a non-linear configuration.

In some embodiments, the at least first layer and the at least second layer are disposed and configured in the pocket via a filling head. In some embodiments, the first API comprises an anticholinergic agent comprising 5-[3-(3-Hydroxyphenoxy)azetidin-1-yl]-5-methyl-2,2-diphenylhexanamide hydrochloride, and the at least second API comprises a bronchodilator comprising salmeterol xinafoate and a corticosteroid comprising fluticasone propionate, as described herein. In some embodiments, the first layer further comprises a first excipient blended with the first API to form a first dry powder, and the second layer further comprises a second excipient blended with the at least second API to form a second dry powder.

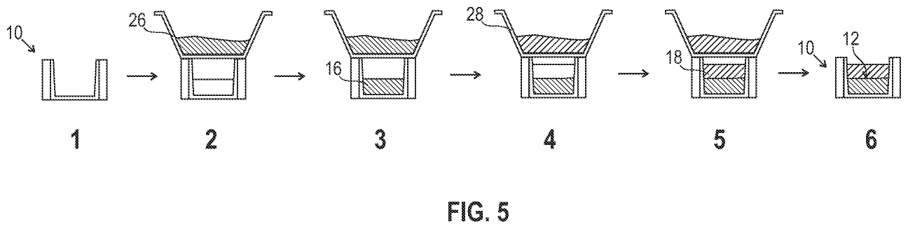

In some embodiments, as shown in FIG. 5, a method for filling a container with a medicament comprising two or more layers each comprising one or more APIs and an excipient is provided. The method comprises (1) providing an empty container 10 such as a dose receptacle, (2) placing the container at a first filling head 26, (3) filling the container with a first layer 16 comprising a first API and a first excipient, (4) placing the container at a second filling head 28, (5) filling the container with a second layer 18 comprising at least a second API and a second excipient, and (6) arriving at a filled or partially filled container comprising the medicament.

In some embodiments, each layer can occupy any percentage of the available container volume provided that the sum percentage of all of the layers is not greater than 100%. In some embodiments, the sum can be mathematically shown as Σ Vₐ, V_{b}, Vₙ≤100%.

In some embodiments, each API remains in its own layer and does not intentionally mix with the other layer. In some embodiments, there is only mixing of the two layers at a boundary layer interface, as described herein.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

The examples below illustrate that two or more APIs can be disposed in fine powder form in a container, where one layer is disposed on the other in a powdered layer filling technique for a medicament that can be used in a dry powder inhaler. One API and an excipient can be disposed in the first layer and a second API and/or third API and an excipient can be disposed in additional layers in a pocket which is suitable for use in conjunction with a dry powder inhaler. In this way, two or more APIs are separate in each layer and substantially mix during inhalation using a dry powder inhaler.

### EXAMPLES

### Example 1

### Comparison of Fine Particle Dose (FPD) Performance of a Blended Combination Product, a Layered Filled Combination Product, and a Baseline Product

The study, as outlined by Example 1, was to assess feasibility of preparing a triple combination product that would deliver similar product performance to that of individual blended combination products without significantly increasing the complexity of the manufacturing process. The two options were to either: 1) layer fill blends of the individual products or; 2) low shear mix blends of the individual products. The individual products were either 0.58% w/w salmeterol xinafoate/4% w/w fluticasone propionate combination blend or 4.8% w/w 5-[3-(3-Hydroxyphenoxy)azetidin-1-yl]-5-methyl-2,2-diphenylhexanamide hydrochloride blend. The option to define a single blend process to deliver similar product performance to that of the individual contributing products by mixing the individual ingredients of the contributing blends is very complex. It is well known by those skilled in the art of dry powder inhalation formulation development that, for example, the mixing scale, blender used, blending parameters (time, speed blade geometry, order of addition of the ingredients and physical characteristics of the ingredients) all influence product performance, and therefore the likelihood of determining a set of parameters which meet similar product performance to that of the individual contributing products may be low and take a long time to determine.

Four products (L1, L2, L3, and T3) were prepared as outline below. Products L1 and L2 were the baseline products for the layer filled combination product L3, and the blended combination product, T3.

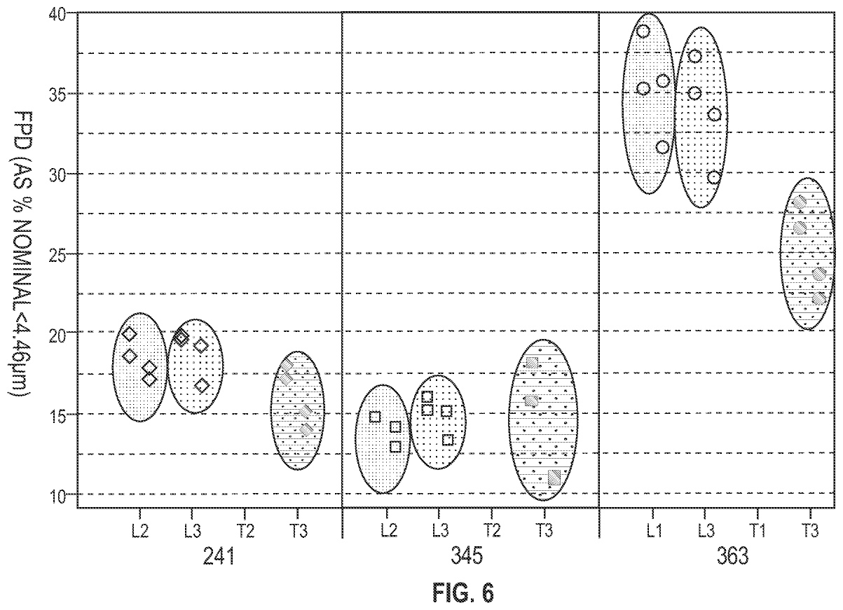

Product L1, as shown in FIG. 6, is a product that has 12.5 mg of lactose monohydrate filled into the first layer of a pocket, and 5.5 mg of 4.8% w/w 5-[3-(3-Hydroxyphenoxy)azetidin-1-yl]-5-methyl-2,2-diphenylhexanamide hydrochloride blend filled into the second layer of the pocket. The blend is made of 4.8% w/w 5-[3-(3-Hydroxyphenoxy)azetidin-1-yl]-5-methyl-2,2-diphenylhexanamide hydrochloride mixed with lactose monohydrate in a Diosna high shear blender. The nominal dose of 5-[3-(3-Hydroxyphenoxy)azetidin-1-yl]-5-methyl-2,2-diphenylhexanamide hydrochloride per pocket is 264 mcg.

Product L2, as shown in FIG. 6, is a product that has 12.5 mg of 0.58% w/w salmeterol xinafoate, 4% w/w fluticasone propionate blend filled into the first layer of a pocket, and 5.5 mg of lactose monohydrate filled into the second layer of the pocket. The blend is made of 0.58% w/w salmeterol xinafoate and 4% w/w fluticasone propionate mixed with lactose monohydrate in a Diosna high shear blender. The nominal doses of salmeterol xinafoate and fluticasone propionate per pocket are 72.5 mcg and 500 mcg, respectively.

Product L3, as shown in FIG. 6, is a product that has 12.5 mg of 0.58% w/w salmeterol xinafoate, 4% w/w fluticasone propionate blend filled into the first layer of a pocket, and 5.5 mg 4.8%w/w 5-[3-(3-Hydroxyphenoxy)azetidin-1-yl]-5-methyl-2,2-diphenylhexanamide hydrochloride blend filled into the second layer of the pocket. The salmeterol xinafoate/fluticasone propionate blend is made of 0.58% w/w salmeterol xinafoate and 4% w/w fluticasone propionate mixed with lactose monohydrate in a Diosna high shear blender. The 5-[3-(3-Hydroxyphenoxy)azetidin-1-yl]-5-methyl-2,2-diphenylhexanamide hydrochloride blend is made of 4.8% w/w 5-[3-(3-Hydroxyphenoxy)azetidin-1-yl]-5-methyl-2,2-diphenylhexanamide hydrochloride mixed with lactose monohydrate in a Diosna high shear blender. The nominal doses of salmeterol xinafoate, fluticasone propionate and 5-[3-(3-Hydroxyphenoxy)azetidin-1-yl]-5-methyl-2,2-diphenylhexanamide hydrochloride per pocket are 72.5 mcg, 500 mcg and 264 mcg, respectively.

Product T3, as shown in FIG. 6, is a product comprising 18 mg of a triple blend of salmeterol xinafoate, fluticasone propionate and 5-[3-(3-Hydroxyphenoxy)azetidin-1-yl]-5-methyl-2,2-diphenylhexanamide hydrochloride. The triple blend is prepared by mixing with a low shear Turbula mixer, a 0.58% w/w salmeterol xinafoate, 4% w/w fluticasone propionate blend with 4.8% w/w 5-[3-(3-Hydroxyphenoxy)azetidin-1-yl]-5-methyl-2,2-diphenylhexanamide hydrochloride blend in the ratio 12.5:5.5. The salmeterol xinafoate/fluticasone propionate blend is made of 0.58% w/w salmeterol xinafoate and 4% w/w fluticasone propionate mixed with lactose monohydrate in a Diosna high shear blender. The 5-[3-(3-Hydroxyphenoxy)azetidin-1-yl]-5-methyl-2,2-diphenylhexanamide hydrochloride blend is made of 4.8% w/w 5-[3-(3-Hydroxyphenoxy)azetidin-1-yl]-5-methyl-2,2-diphenylhexanamide hydrochloride mixed with lactose monohydrate in a Diosna high shear blender. The nominal doses of salmeterol xinafoate, fluticasone propionate and 5-[3-(3-Hydroxyphenoxy)azetidin-1-yl]-5-methyl-2,2-diphenylhexanamide hydrochloride per pocket are 72.5 mcg, 500 mcg and 264 mcg, respectively.

TABLE 1
Summary of Formulations used in Example 1
for the Data Presented in FIG. 6 Product

| Product | Layer 1 | Layer 2 |
|---|---|---|
| L1 (Baseline | 12.5 mg of lactose | 5.5 mg of 4.8% w/w 5-[3-(3- |
| for L3, T3, | monohydrate | Hydroxyphenoxy)azetidin- |
| and drug | | 1-yl]-5-methyl-2,2- |
| 363) | | diphenylhexanamide |
| | | hydrochloride blend |

Nominal dose per pocket: 264 mcg of 5-[3-
(3-Hydroxyphenoxy)azetidin-1-yl]-5-methyl-
2,2-diphenylhexanamide hydrochloride

| | | |
|---|---|---|
| L2 (Baseline | 12.5 mg of 0.58% w/w | 5.5 mg of lactose monohydrate |
| for L3, T3, | salmeterol xinafoate, | |
| drug 241 | 4% w/w fluticasone | |
| and 345) | propionate blend | |

Nominal dose per pocket: 72.5 mcg salmeterol
xinafoate, 500 mcg fluticasone propionate

| | | |
|---|---|---|
| L3 (layer | 12.5 mg of 0.58% w/w | 5.5 mg of 4.3% w/w 5-[3-(3- |
| filled | salmeterol xinafoate, | Hydroxyphenoxy)azetidin- |
| combination | 4% w/w fluticasone | 1-yl]-5-methyl-2,2- |
| of drug | propionate blend | diphenylhexanamide |
| 363, 241, | | hydrochloride blend |

| | |
|---|---|
| and 345) | Nominal dose per pocket: 72.5 mcg salmeterol |
| | xinafoate, 500 mcg fluticasone propionate 236 mcg |
| | nominal dose of 5-[3-(3-Hydroxyphenoxy)azetidin- |
| | 1-yl]-5-methyl-2,2-diphenylhexanamide |
| | hydrochloride |

| | | |
|---|---|---|
| T3 (blend | 18 mg of a 58% w/w | N/A |
| combination | salmeterol xinafoate, | |
| of drug | 4% w/w fluticasone | |
| 363, 241, | propionate blend mixed | |
| and 345) | with 4.8% w/w 5-[3-(3- | |
| | Hydroxyphenoxy)azetidin- | |
| | 1-yl]-5-methyl-2,2- | |
| | diphenylhexanamide | |
| | hydrochloride blend in | |
| | the ratio 12.5:5.5 | |

Nominal dose per pocket: 72.5 mcg salmeterol
xinafoate, 500 mcg fluticasone propionate 264 mcg
5-[3-(3-Hydroxyphenoxy)azetidin-1-yl]-5-methyl-
2,2-diphenylhexanamide hydrochloride

363 = carboxamide HCL (MGR002)
241 = fluticasone propionate
345 = salmeterol xinafoate
241/345 = combination product of fluticasone propionate and salmeterol xinafoate (MGR001)

The results plotted in FIG. 6 indicated that a layer filled combination product L3 delivers comparable performance to baseline products L1 and L2. The blended combination product T3 did not indicate similar fine particle dose across all three drugs to the baseline product. Drug 363 had a much lower FPD, drug 241 had a slightly lowered FPD and drug 345 showed more variability.

### Example 2

### Comparison of Product Layering Order

The study, as outlined by Example 2, was to determine filling layer order of two blends based on the control of the fill weight. The products in the study were at a fixed strength of 0.58% w/w salmeterol xinafoate/2% w/w fluticasone propionate combination blend layer, filled with either a low strength 1.1% w/w 5-[3-(3-Hydroxyphenoxy)azetidin-1-yl]-5-methyl-2,2-diphenylhexanamide hydrochloride (equivalent to 1% w/w as the free base) blend or a high strength 8.3% w/w 5-[3-(3-Hydroxyphenoxy)azetidin-1-yl]-5-methyl-2,2-diphenylhexanamide hydrochloride (equivalent to 7.5% w/w as the free base) blend.

The salmeterol xinafoate/fluticasone propionate blend is made of 0.58% w/w salmeterol xinafoate and 2% w/w fluticasone propionate mixed with lactose monohydrate in a Diosna high shear blender. The 5-[3-(3-Hydroxyphenoxy)azetidin-1-yl]-5-methyl-2,2-diphenylhexanamide hydrochloride blend is made with either 1.1% w/w or 8.3% w/w 5-[3-(3-Hydroxyphenoxy)azetidin-1-yl]-5-methyl-2,2-diphenylhexanamide hydrochloride mixed with lactose monohydrate in a Picomix high shear blender. The blends were filled to make the product combinations detailed in Table 2.

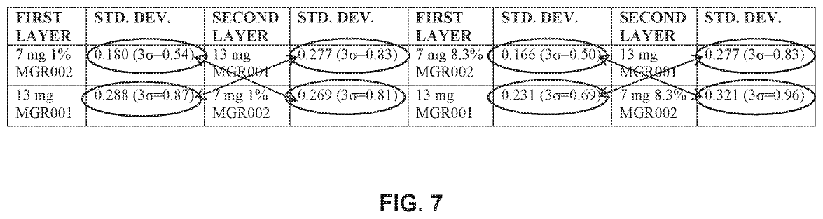

TABLE 2
Summary of Formulations used in Example 2 for the Data Presented in FIG. 7

| Product | Layer 1 | Layer 2 |
|---|---|---|
| Row 1 | 7 mg of 1.1% w/w 5-[3-(3- | 13 mg of 12.5 mg of 0.58% w/w |
| (columns | Hydroxyphenoxy)azetidin-1-yl]- | salmeterol xinafoate, 2% w/w |
| 1-4) | 5-methyl-2,2- | fluticasone propionate blend |
| | diphenylhexanamide | |
| | hydrochloride blend (MGR002) | |
| Row 1 | 7 mg of 8.3% w/w 5-[3-(3- | 13 mg of 12.5 mg of 0.58% w/w |
| (columns | Hydroxyphenoxy)azetidin-1-yl]- | salmeterol xinafoate, 2% w/w |
| 5-8) | 5-methyl-2,2- | fluticasone propionate blend |
| | diphenylhexanamide | |
| | hydrochloride blend | |
| Row 2 | 13 mg of 12.5 mg of 0.58% w/w | 7 mg of 1.1% w/w 5-[3-(3- |
| (columns | salmeterol xinafoate, 2% w/w | Hydroxyphenoxy)azetidin-1-yl]-5- |
| 1-4) | fluticasone propionate blend | methyl-2,2-diphenylhexanamide |
| | | hydrochloride blend |
| Row 2 | 13 mg of 12.5 mg of 0.58% w/w | 7 mg of 8.3% w/w 5-[3-(3- |
| (columns | salmeterol xinafoate, 2% w/w | Hydroxyphenoxy)azetidin-1-yl]-5- |
| 5-8) | fluticasone propionate blend | methyl-2,2-diphenylhexanamide |
| | | hydrochloride blend |

Early product performance data suggested that the layer order did not matter. The results illustrated in FIG. 7 indicated that from a fill weight perspective, it is beneficial to fill the lower fill weight product first to reduce the variability observed (e.g., standard deviation). When 13 mg of salmeterol xinafoate combined with fluticasone propionate and lactose monohydrate is filled in either the first or second layer, the variability in fill weight remains constant (e.g., shown by the oval in column 2, row 3 and column 4, row 2, and column 6, row 3 and column 8, row 2 of FIG. 7). When 7 mg of 5-[3-(3-Hydroxyphenoxy)azetidin-1-yl]-5-methyl-2,2-diphenylhexanamide hydrochloride and lactose monohydrate is filled in the first layer (e.g., shown by the oval in row 2 and columns 2 and 6 of FIG. 7), the variability is lower than when filled in the second layer (e.g., shown by the oval in row 3 columns 4 and 8 of FIG. 7). When 7 mg of the 5-[3-(3-Hydroxyphenoxy)azetidin-1-yl]-5-methyl-2,2-diphenylhexanamide hydrochloride and lactose monohydrate is filled first followed by 13 mg of salmeterol xinafoate combined with fluticasone propionate and lactose monohydrate, the overall total fill weight variability is also reduced.

### Example 3

### Stability Study

In the examples, MGR001 refers to a dry powder for inhalation in a CRC749 dry powder inhaler device, wherein the actives are fluticasone propionate and salmeterol xinafoate in a ratio of 250 μg/50 μg. CRC749 dry powder inhaler has been described in U.S. Pat. Nos. 9,399,103 and/or 9,561,336, incorporated herein by reference as if set forth in full.

MGR002 refers to a dry powder formulation for inhalation (441 μg) in a CRC749 dry powder inhaler device, wherein the active ingredient is 5-[3-(3-Hydroxyphenoxy) azetidin-1-yl]-5-methyl-2,2-diphenylhexanamide, a carboxamide compound of formula I. In some embodiments, MGR002 can be a 7%w/w blend of 5-[3-(3-Hydroxyphenoxy) azetidin-1-yl]-5-methyl-2,2-diphenylhexanamide in lactose monohydrate.

MGR003 refers to a dry powder formulation for inhalation in a CRC749 dry powder inhaler device, wherein the actives are a fluticasone propionate, salmeterol xinafoate and carboxamide hydrochloride of formula II in a ratio of 250 μg/50 μg/441 μg. In some embodiments, the MGR003 is a layered fill product of MGR002 and MGR001.

In this example, MGR002 is 5-[3-(3-Hydroxyphenoxy) azetidin-1-yl]-5-methyl-2,2-diphenylhexanamide (the carboxamide compound) and lactose monohydrate and a MGR003, a bi-layered medicament comprising a first layer comprising 5-[3-(3-Hydroxyphenoxy)azetidin-1-yl]-5-methyl-2,2-diphenylhexanamide hydrochloride, and a second layer comprising salmeterol xinafoate and fluticasone propionate (MGR001) were studied for stability. The 5-[3-(3-Hydroxyphenoxy) azetidin-1-yl]-5-methyl-2,2-diphenylhexanamide was at 7% w/w blend in 60 μm lactose monohydrate LH200 and was achieved by a 600 rpm and 10 minute blending process at 1.6 kg scale, in a 4 L Diosna bowl. All batches utilized in this study were filled at a set humidity range of 45% RH±5% and temperature of 20° C.±2° C. Fill weight targets used in this stability study are detailed in Tables 3 and 4 below.

TABLE 3
Set up fill weight targets for MGR003

| Set-up | Target (mg) | Range (mg) |
|---|---|---|
| Mean pocket fill weight (5-[3-(3- | 7.00 ± 2.5% | 6.83-7.18 |
| Hydroxyphenoxy)azetidin-1-yl]-5- | | |
| methyl-2,2-diphenylhexanamide | | |
| hydrochloride, first layer) | | |
| Individual pocket weight (5-[3-(3- | 7.00 ± 10.0% | 6.30-7.70 |
| Hydroxyphenoxy)azetidin-1-yl]-5- | | |
| methyl-2,2-diphenylhexanamide | | |
| hydrochloride, first layer) | | |
| % RSD (5-[3-(3-Hydroxyphenoxy)azetidin- | <3.50 | n/a |
| 1-yl]-5-methyl-2,2- [symbol] | | |
| Mean pocket fill weight (first layer + | 20.00 ± 1.0% | 19.80-20.20 |
| fluticasone propionate and salmeterol | | |
| xinafoate dry powder blend, second layer) | | |
| Individual pocket weight (first layer + | 20.00 ± 5.0% | 19.00-21.00 |
| second layer) | | |
| % RSD (first layer + second layer) | ≤3.50 | n/a |

RSD refers to relative standard deviation [symbol]
indicates data missing or illegible when filed

TABLE 4
Manufacturing fill weight targets for MGR003

| Manufacturing limits | Target (mg) | Range (mg) |
|---|---|---|
| Mean pocket fill weight (5-[3-(3- | 7.00 ± 5.0% | 6.65-7.35 |
| Hydroxyphenoxy)azetidin-1-yl]-5- | | |
| methyl-2,2-diphenylhexanamide | | |
| hydrochloride, first layer) | | |
| Individual pocket weight (5-[3-(3- | 7.00 ± 10.0% | 6.30-7.70 |
| Hydroxyphenoxy)azetidin-1-yl]-5- | | |
| methyl-2,2-diphenylhexanamide | | |
| hydrochloride, first layer) | | |
| % RSD (5-[3-(3-Hydroxyphenoxy)azetidin- | <3.50 | n/a |
| 1-yl]-5-methyl-2,2-diphenylhexanamide | | |
| hydrochloride, first layer) | | |
| Mean pocket fill weight (first layer + | 20.00 ± 2.0% | 19.60-20.40 |
| fluticasone propionate and salmeterol | | |
| xinafoate dry powder blend, second layer) | | |
| Individual pocket weight (first layer + | 20.00 ± 6.0% | 18.80-21.20 |
| second layer) | | |
| % RSD (first layer + second layer) | ≤3.50 | n/a |

In Tables 3 and 4 above, MGR002 used in the first layer was 7% w/w and contained in addition to 5-[3-(3-Hydroxyphenoxy)azetidin-1-yl]-5-methyl-2,2-diphenylhexanamide hydrochloride, also 60 μm LH200 or 80 μm LH200 lactose monohydrate. For the second layer of fluticasone propionate and salmeterol xinafoate powder blend, MGR001 contained a 2% w/w/0.58% w/w blend of each active ingredient. By combining a first layer of MGR002 with a second layer of MGR001 in the pockets of a disc, a batch of MGR003 is obtained which contains 250 μg/50 μg/490 μg of fluticasone propionate and salmeterol xinafoate and 5-[3-(3-Hydroxyphenoxy)azetidin-1-yl]-5-methyl-2,2-diphenylhexanamide hydrochloride of formula II.

### Aerosolization Performance

The same batches for MGR002 and MGR003 were utilized in obtaining emitted data used to obtain aerosolization performance information. Table 5 below presents the results and equivalence criteria, respectively for 5-[3-(3-Hydroxyphenoxy)azetidin-1-yl]-5-methyl-2,2-diphenylhexanamide hydrochloride of formula II, fluticasone propionate and salmeterol.

TABLE 5
Equivalence criteria for TIR and FPM for MGR001, MGR002 and MGR003,
Hydroxyphenoxy)azetidin-1-yl]-5-methyl-2,2-diphenylhexanamide
hydrochloride, salmeterol and fluticasone propionate

| 5-[3-(3- | Ratio of | | | |
|---|---|---|---|---|
| Hydroxyphenoxy)azetidin-1-yl]- | geometric means | | | |
| 5-methyl-2,2-diphenylhexanamide | (MGR002 divided | | | Pass/fail |
| hydrochloride | by MGR003) | Lower 90% CI | Upper 90% CI | (0.90-1.11) |
| TIR | 0.97 | 0.95 | 0.99 | Pass |
| FPM | 0.95 | 0.91 | 1.00 | Pass |
| | Ratio of | | | |
| | geometric means | | | |
| | (MGR001 divided | | | Pass/fail |
| Salmeterol | by MGR003) | Lower 90% CI | Upper 90% CI | (0.90-1.11) |
| TIR | 0.97 | 0.96 | 0.98 | Pass |
| FPM | 1.01 | 0.97 | 1.05 | Pass |
| | Ratio of | | | |
| | geometric means | | | |
| | (MGR001 divided | | | Pass/fail |
| Fluticasone propionate | by MGR003) | Lower 90% CI | Upper 90% CI | (0.90-1.11) |
| TIR | 0.96 | 0.93 | 0.98 | Pass |
| FPM | 0.99 | 0.95 | 1.03 | Pass |

The mean particle mass (FPM) and mean total impactor recovery (TIR) from a next generation impactor (NGI) for each batch were equivalent, based on the fact that the upper/lower 90% confidence interval of the ratio of the geometric mean, rested between 0.90 and 1.11. Table 3 also shows the results and equivalence criteria for salmeterol xinofoate and fluticasone propionate for batches MGR001 and MGR003. The fluticasone propionate and salmeterol xinafoate components of the MGR003 drug product also met the same performance targets as the MGR001 250/50 drug product.

Testing of both MGR002 and MGR003 products between an initial time and 6 months at 25° C./60% RH, 30° C./60% RH and 40° C./75% RH yielded a mean FPF % TIR values in a range from about 31 to about 37 mean data labels. FPM values between from about 110 μg to about 160 μg mean data labels were obtained without any observable trends on stability. Both the MGR002 and MGR003 equivalence study demonstrated that the FPM of 5-[3-(3-Hydroxyphenoxy)azetidin-1-yl]-5-methyl-2,2-diphenylhexanamide hydrochloride compound is equivalent for both products, using the IND stability batches and also MGR001 is equivalent to MGR003 for salmeterol (as xinafoate) and fluticasone propionate.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

1. A medicament for inhalation, the medicament comprising at least a first layer comprising a first active pharmaceutical ingredient in powder form and at least a second layer comprising at least a second active pharmaceutical ingredient in powder form, the second layer disposed on the first layer.

2. The medicament of claim 1, wherein the at least first layer further comprises a first excipient blended with the first active pharmaceutical ingredient to form a first dry powder and the at least second layer further comprises a second excipient blended with the at least second active pharmaceutical ingredient to form a second dry powder.

3. The medicament of claim 2, wherein the first and second excipients are independently selected from the group consisting of: glucose, arabinose, lactose, sucrose, maltose, dextrans, magnesium stearate, leucine, isoleucine, lysine, valine, methionine, phenylalanine, and combinations thereof.

4. The medicament of claim 1, wherein the at least first layer and the at least second layer comprise micronized powder.

5. The medicament of claim 1, wherein: (i) the at least second layer is disposed on the at least first layer in a linear configuration; (ii) the at least second layer is disposed on the at least first layer in a non-linear configuration; or (iii) the first active pharmaceutical ingredient and the at least second active pharmaceutical ingredient are different active pharmaceutical ingredients.

6. The medicament of claim 1, wherein the at least first layer and the at least second layer mix at a boundary layer interface in an amount from about 0.01 to about 10 wt. %.

7. The medicament of claim 1, wherein: (i) the first active pharmaceutical ingredient comprises an anticholinergic agent and the at least second active pharmaceutical ingredient comprises a bronchodilator; (ii) the first active pharmaceutical ingredient comprises an anticholinergic agent and the at least second active pharmaceutical ingredient comprises a bronchodilator and the medicament comprises a third active pharmaceutical ingredient comprising a corticosteroid; or
(iii) the first active pharmaceutical ingredient comprises an anticholinergic agent in the first layer and the at least second active pharmaceutical ingredient comprises a bronchodilator and a corticosteroid in the second layer.

8. The medicament of claim 7, wherein the anticholinergic agent comprises 5-[3-(3-Hydroxyphenoxy)azetidin-1-yl]-5-methyl-2,2-diphenylhexanamide hydrochloride, the bronchodilator comprises salmeterol xinafoate, and the corticosteroid comprises fluticasone propionate.

9. The medicament of claim 7, wherein the anticholinergic agent comprises the structure of

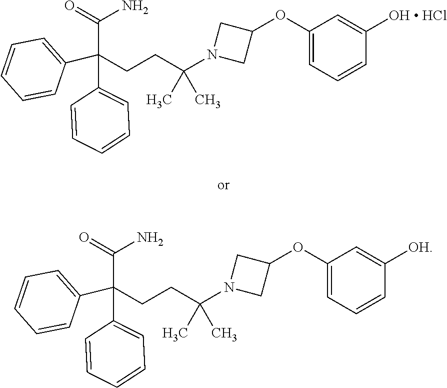

10. The medicament of claim 8, wherein the 5-[3-(3-Hydroxyphenoxy)azetidin-1-yl]-5-methyl-2,2-diphenylhexanamide hydrochloride is in a dose of about 40 to about 800 mcg, the salmeterol xinafoate is in a dose of about 72.5 mcg, and the fluticasone propionate is in a dose of about 100 to about 500 mcg.

11. The medicament of claim 8, wherein the 5-[3-(3-Hydroxyphenoxy)azetidin-1-yl]-5-methyl-2,2-diphenylhexanamide hydrochloride has an average particle size of from about 1 μm to about 10 μm, the salmeterol xinafoate has an average particle size of from about 1 μm to about 10 μm, and the fluticasone propionate has an average particle size from about 1 μm to about 3 μm.

12. A container for dispensing a medicament for inhalation, the container having one or more doses of the medicament, and the medicament comprising at least a first layer comprising a first active pharmaceutical ingredient in powder form and at least a second layer comprising at least a second active pharmaceutical ingredient in powder form, the second layer disposed on the first layer.

13. The container of claim 12, wherein the medicament is used for oral inhalation.

14. The container of claim 12, wherein the container comprises a pocket containing a dose of the medicament and a covering disposed on the container, the covering configured for piercing engagement such that the dose of the medicament can be dispensed from the container.

15. The container of claim 12, wherein the at least first layer and the at least second layer mix at a boundary layer interface in an amount from about 0.01 to about 10 wt. %.

16. The container of claim 14, wherein: (i) the first active pharmaceutical ingredient comprises an anticholinergic agent comprising 5-[3-(3-Hydroxyphenoxy)azetidin-1-yl]-5-methyl-2,2-diphenylhexanamide hydrochloride, the at least second active pharmaceutical ingredient comprises a bronchodilator comprising salmeterol xinafoate; (ii) the first active pharmaceutical ingredient comprises an anticholinergic agent comprising 5-[3-(3-Hydroxyphenoxy)azetidin-1-yl]-5-methyl-2,2-diphenylhexanamide hydrochloride, the at least second active pharmaceutical ingredient comprises a bronchodilator comprising salmeterol xinafoate and a corticosteroid comprising fluticasone propionate; or (iii) the first active pharmaceutical ingredient comprises an anticholinergic agent comprising 5-[3-(3-Hydroxyphenoxy)azetidin-1-yl]-5-methyl-2,2-diphenylhexanamide hydrochloride, the at least second active pharmaceutical ingredient comprises a bronchodilator comprising salmeterol xinafoate and the medicament further comprises a third layer comprising a third active pharmaceutical ingredient comprising a corticosteroid comprising fluticasone propionate.

17. The container of claim 16, wherein the 5-[3-(3-Hydroxyphenoxy)azetidin-1-yl]-5-methyl-2,2-diphenylhexanamide hydrochloride is in a dose of about 40 to about 800 mcg, the salmeterol xinafoate is in a dose of about 72.5 mcg, and the fluticasone propionate is in a dose of about 100 to about 500 mcg.

18. The container of claim 12, wherein: (i) the medicament has a density of about 0.6 to about 0.9 mg/μl; or (ii) the medicament comprises an excipient comprising glucose, arabinose, lactose, mannitol, sucrose, maltose, dextrans, magnesium stearate, leucine, isoleucine, lysine, valine, methionine, phenylalanine, or a combination thereof.

19. The container of claim 14, wherein the pocket comprises a fill weight of the first active pharmaceutical ingredient and a first excipient that is in an amount of about 3 to about 9 mg, and a fill weight of the at least second active pharmaceutical ingredient and a second excipient that is in an amount of about 8 to about 13 mg.

20. The container of claim 14, wherein the pocket comprises a fill weight of the first active pharmaceutical ingredient and a first excipient that is in an amount of about 7 mg, a fill weight of the at least second active pharmaceutical ingredient and a second excipient that is in an amount of about 7 mg and a fill weight of a third active pharmaceutical ingredient and a third excipient in an amount of about 7 mg.

21.-25. (canceled)
